(19) United States
(12) Patent Application Publication
(10) Pub. No.: US 2025/0345625 A1
(43) Pub. Date: Nov. 13, 2025

(54) PHOTOTHERAPEUTIC ILLUMINATION DEVICES WITH DATA CAPTURING

(71) Applicant: KNOW Bio, LLC, Morrisville, NC (US)

(72) Inventors: Antony Paul van de Ven, Nong Kae (TH); F. Neal Hunter, Morrisville, NC (US)

(21) Appl. No.: 19/202,796

(22) Filed: May 8, 2025

Related U.S. Application Data
(60) Provisional application No. 63/646,002, filed on May 13, 2024.

(51) Int. Cl. A61N 5/06; H04N 23/56; H04N 23/695; H04N 23/90

(52) U.S. Cl. CPC A61N 5/0617; A61N 2005/0626; A61N 2005/0647; A61N 2005/0652; A61N 2005/0659; A61N 2005/0663; A61N 2005/0665; H04N 23/56; H04N 23/695; H04N 23/90

(57) ABSTRACT

Illumination devices for directing light on tissue to induce one or more biological effects and more particularly phototherapeutic illumination devices with data capturing capabilities are disclosed. Illumination devices for phototherapy include integrated data capturing capabilities while also being capable of delivering phototherapeutic treatments to the scalp. Data capturing capabilities include imaging capabilities where one or more integrated cameras within an illumination device are capable of capturing images of the scalp before, during, and/or after phototherapy. In certain embodiments, data capturing capabilities include the ability to map larger regions of the scalp or the entire scalp. Related systems are disclosed capable of accessing captured data from illumination devices, identifying and characterizing one or more features in the data, such as hair, hair density, and hair follicles, as well as other conditions of the hair and/or scalp.

### RELATED APPLICATIONS

This application claims the benefit of provisional patent application Ser. No. 63/646,002, filed May 13, 2024, the disclosure of which is hereby incorporated herein by reference in its entirety.

### FIELD OF THE DISCLOSURE

The present disclosure relates generally to illumination devices for directing light on tissue to induce one or more biological effects and more particularly to phototherapeutic illumination devices with data capturing capabilities.

### BACKGROUND

Androgenetic alopecia is a common form of hair loss in both men and women. Although risk factors contributing to this condition are still being studied, researchers have determined that androgenetic alopecia is related to hormones called androgens, and particularly to an androgen called dihydrotestosterone. Increased levels of androgens in hair follicles can lead to a shorter cycle of hair growth, as well as the growth of shorter and thinner strands of hair. Early stages of hair loss can be slowed or reversed with medication. Other treatment options include tretinoin combined with minoxidil, ketoconazole shampoo, and spironolactone. Advanced cases of hair loss may be resistant or unresponsive to pharmaceutical therapy. A number of patients elect to undergo surgical hair transplantation.

Various phototherapy devices for addressing androgenetic alopecia have been developed. The term “phototherapy” relates to the therapeutic use of light. Without necessarily being directed to treatment of hair loss, various light therapies (e.g., including low level light therapy (LLLT) and photodynamic therapy (PDT)) have been publicly reported or claimed to provide various health related medical benefits-including, but not limited to: treating skin or tissue inflammation; promoting tissue or skin healing or rejuvenation; enhancing wound healing; pain management; reducing wrinkles, scars, stretch marks, varicose veins, and spider veins; enhancing mood; treating microbial infections; treating hyperbilirubinemia; and treating various oncological and non-oncological diseases or disorders.

Various mechanisms by which phototherapy has been suggested to provide therapeutic benefits include: increasing circulation (e.g., by increasing formation of new capillaries); stimulating the production of collagen; stimulating the release of adenosine triphosphate (ATP); enhancing porphyrin production; reducing excitability of nervous system tissues; stimulating fibroblast activity; increasing phagocytosis; inducing thermal effects; stimulating tissue granulation and connective tissue projections; reducing inflammation; and stimulating acetylcholine release. Phototherapy has also been suggested to stimulate cells to generate nitric oxide. Various biological functions attributed to nitric oxide include roles as signaling messenger, cytotoxin, antiapoptotic agent, antioxidant, and regulator of microcirculation. Nitric oxide is recognized to relax vascular smooth muscles, dilate blood vessels, inhibit aggregation of platelets, and modulate T cell-mediated immune response. Nitric oxide is produced by multiple cell types in skin and is formed by the conversion of the amino acid L-arginine to L-citrulline and nitric oxide, mediated by the enzymatic action of nitric oxide synthases (NOSs).

Existing phototherapy devices for addressing androgenetic alopecia have limitations that affect their utility. Rigid helmet-type phototherapy devices may be uncomfortable and unsightly for many users, and such devices may be cumbersome to manufacture. Providing substantially uniform and/or uninterrupted coverage over an entire area to be treated may also be challenging for conventional phototherapy helmets, caps, and combs (e.g., as they require user movement and compliance). Thermal management may also be a concern for conventional phototherapy helmets and caps.

The art continues to seek improved phototherapy devices providing desirable illumination characteristics and capable of overcoming challenges associated with conventional phototherapy devices.

### SUMMARY

The present disclosure relates generally to illumination devices for directing light on tissue to induce one or more biological effects and more particularly to phototherapeutic illumination devices with data capturing capabilities. Illumination devices for phototherapy include integrated data capturing capabilities while also being capable of delivering phototherapeutic treatments to the scalp. Data capturing capabilities include imaging capabilities where one or more integrated cameras within an illumination device are capable of capturing images of the scalp before, during, and/or after phototherapy. In certain embodiments, data capturing capabilities include the ability to map larger regions of the scalp or even the entire scalp. Related systems are disclosed capable of accessing captured data from illumination devices, identifying and characterizing one or more features in the data, such as hair, hair density, and hair follicles, as well as other conditions of the hair and/or scalp.

In one aspect, a phototherapy device for delivering light emissions to a scalp of a patient comprises: a flexible substrate comprising a proximal surface and a distal surface that is opposite the proximal surface, the flexible substate being configured for positioning along a scalp of a user such that the proximal surface is closer to the scalp than the distal surface; an array of light-emitting devices on the proximal surface; driver circuitry configured to drive the array of light-emitting devices; and a data capture device on the proximal surface of the flexible substrate, the data capture device configured to capture data of the scalp. The phototherapy device may further a proximity sensor on the proximal surface of the flexible substrate. In certain embodiments, the data capture device is configured to initiate capturing the data based on a signal from the proximity sensor. In certain embodiments, the data comprises a progression of images configured to be captured as the camera is moved closer to the scalp.

In certain embodiments, the data capture device is a camera and the data comprises one or more images of the scalp. The phototherapy device may further comprise a flexible optic associated with the camera, wherein the flexible optic is configured to compress when the flexible optic contacts the scalp. In certain embodiments, the camera is configured to initiate image capturing when the flexible optic contacts the scalp. In certain embodiments, the data capture device is one of a plurality of data capture devices on the proximal surface of the flexible substrate. In certain embodiments, the data capture device is a first camera of a plurality of cameras, the first camera is configured to capture a near field image of the scalp during use, and a second camera of the plurality of cameras is configured to capture a far field image of the scalp during use.

The phototherapy device may further comprise a parting structure configured to create a part line in hair of the user. In certain embodiments, the parting structure comprises a fan. In certain embodiments, the parting structure comprises a comb feature. The phototherapy device may further comprise a scalp covering configured to conform to the scalp. In certain embodiments, the scalp covering comprises a mesh structure to permit passage of light from the array of light-emitting devices. In certain embodiments, the scalp covering comprises a window within a field of view of the data capture device. In certain embodiments, the window comprises an antireflective material.

In another aspect, a phototherapy device for delivering light emissions to a scalp of a patient comprises: an array of light-emitting devices; driver circuitry configured to drive the array of light-emitting devices; and a data capture device configured to capture data of the scalp of a user, a position of the camera being adjustable relative to a position of the array of light-emitting devices. In certain embodiments, the position of the data capture device is configured for manual adjustment. In certain embodiments, the position of the data capture device is configured for automated adjustment. The phototherapy device may further comprise a flexible substrate comprising a proximal surface and a distal surface that is opposite the proximal surface, the flexible substate being configured for positioning along the scalp such that the proximal surface is closer to the scalp than the distal surface, wherein the array of light-emitting devices is on the proximal surface. The phototherapy device may further comprise a movement channel for the data capture device, the movement channel being formed through the flexible substrate. The phototherapy may further comprise: a cap comprising a cap proximal surface and a cap distal surface that is opposite the cap proximal surface, the cap proximal surface being positioned closer to the flexible substrate than the cap distal surface; and a focus adjustment structure configured to provide focus control of the data capture device, the focus adjustment structure being accessible from the cap distal surface. The phototherapy device may further comprise a parting structure configured to create a part line in hair of the user.

In another aspect, a phototherapy device for delivering light emissions to a scalp of a patient comprises: an array of light-emitting devices; driver circuitry configured to drive the array of light-emitting devices; and a camera configured to capture one or more images of the scalp of a user via reflection from a mirror. The phototherapy device may further comprise a cap with an opening positioned to expose a portion of the scalp, wherein the mirror is positioned proximate the opening. In certain embodiments, the mirror and camera reside within a housing on the cap. In certain embodiments, the mirror is deformable. In certain embodiments, the mirror forms a concave surface relative to the camera. The phototherapy device may further comprise a parting structure configured to create a part line in hair of the user.

In another aspect, a method comprises: accessing image data related to an image of an area of a scalp; enhancing the image data by increasing contrast of the image, followed by grayscale conversion and image conversion; identifying hair from the enhanced image data based on data from previously accessed data from an image library; and generating a density of the hair in the image. The method may further comprise sending a treatment protocol to a phototherapy device based on the density of the hair in the image. In certain embodiments, identifying the hair and generating the density of the hair is provided by an artificial intelligence classifier that accesses the image data and the image library. The method may further comprise generating a hair density map of the scalp based on a plurality of images of different areas of the scalp.

In another aspect, a system comprises: a phototherapy device comprising a light-emitting device and a data capture device, the phototherapy device configured to capture data of a scalp of a user; and a server in communication with the phototherapy device via a network, wherein the server is configured to access the data of the scalp, identify hair in the data of the scalp, and generate a density of the hair in the data. In certain embodiments, the server comprises an artificial intelligence classifier configured to identify the hair and generate the density of the hair in the data based on other data accessed from an image library. In certain embodiments, the server is configured to provide a treatment protocol to the phototherapy device based on the density of the hair in the data. In certain embodiments, the data of the scalp is a plurality of images of the scalp, and the server is configured to generate a hair density map of the scalp based on the plurality of images.

In another aspect, a phototherapy device for delivering light emissions to a scalp of a patient comprises: one or more first light-emitting devices configure to provide phototherapeutic light to the scalp; a data capture device configured to capture data of the scalp; and a scalp covering positioned between the one or more light-emitting devices and the scalp. In certain embodiments, the scalp covering is configured to conform to the scalp and hold down hair when the phototherapeutic light is provided to the scalp or when the data of the scalp is captured. The phototherapy device may further comprise one or more additional light-emitting devices configured to provide general illumination to one or more portions of the scalp when the data of the scalp is captured. The phototherapy device may further comprise a reflector positioned to reflect light from the one or more additional light-emitting devices toward the one or more portions of the scalp. In certain embodiments, the reflector forms a structure with a first opening proximate the data capture device and a second opening proximate the scalp. In certain embodiments, the one or more additional light-emitting devices are mounted on an inner sidewall of the reflector between the first opening and the second opening.

In another aspect, a phototherapy device for delivering light emissions to a scalp of a patient comprises: one or more light-emitting devices configured to provide phototherapeutic light to the scalp; a first data capture device configured to capture first data of the scalp; and a second data capture device configured to capture second data of the scalp, the second data covering a greater area of the scalp than the first data. In certain embodiments, the first data capture device is a first camera and the second data capture device is a second camera. The phototherapy device may further comprise first additional light-emitting devices configured to provided imaging light to the scalp for the first camera. The phototherapy device may further comprise second additional light-emitting devices configured to provide imaging light to the scalp for the second camera. In certain embodiments, a field of view of the second camera overlaps at least a portion of a field of view of the first camera. The phototherapy device may further comprise a first proximity sensor configured to initiate the first data capture device to capture the first data when the phototherapy device is positioned on the scalp. The phototherapy device may further comprise a second proximity sensor configured to initiate the second data capture device to capture the second data when the phototherapy device is positioned in a spaced relationship over the scalp.

In another aspect, any of the foregoing aspects individually or together, and/or various separate aspects and features as described herein, may be combined for additional advantage. Any of the various features and elements as disclosed herein may be combined with one or more other disclosed features and elements unless indicated to the contrary herein.

Those skilled in the art will appreciate the scope of the present disclosure and realize additional aspects thereof after reading the following detailed description of the preferred embodiments in association with the accompanying drawing figures.

### BRIEF DESCRIPTION OF THE DRAWING FIGURES

The accompanying drawing figures incorporated in and forming a part of this specification illustrate several aspects of the disclosure, and together with the description serve to explain the principles of the disclosure.

FIG. 1A is a perspective view of an illumination device embodying a wearable cap for delivering light to the scalp of a patient.

FIG. 1B is a bottom view of the illumination device of FIG. 1A illustrating a view within a concavity of the illumination device.

FIG. 1C is an exploded view of the illumination device of FIGS. 1A and 1B.

FIG. 2A is a view of the illumination device of FIGS. 1A to 1C at an initial step for positioning the illumination device on the scalp of the user.

FIG. 2B is a representation of a first image of the scalp taken when the illumination device is positioned according to FIG. 2A.

FIG. 2C is a view of the illumination device of FIG. 2A at a subsequent step where the illumination device is positioned closer to the scalp, but not in a position to begin light treatment.

FIG. 2D is a representation of a second image of the scalp taken when the illumination device is positioned according to FIG. 2C.

FIG. 2E is a view of the illumination device at a subsequent step to FIG. 2C where the illumination device is positioned proximate the scalp and in a position to begin light treatment.

FIG. 2F is a representation of a third image of the scalp taken when the illumination device is positioned according to FIG. 2E.

FIG. 3A is a top perspective view of an illumination device that is similar to the illumination device of FIGS. 1A to 1C for embodiments where a position of the camera is adjustable.

FIG. 3B is a front view of the illumination device of FIG. 3A.

FIG. 4A is a front view of an illumination device that is similar to the illumination device of FIGS. 3A and 3B for embodiments that include an array of cameras.

FIG. 4B is a top view of the illumination device of FIG. 4A.

FIG. 5 is a front view of an illumination device that is similar to the illumination device of FIGS. 4A and 4B for embodiments that include different cameras that capture different fields of view.

FIG. 6 is a side view of an illumination device that is similar to the illumination device of FIGS. 4A and 4B for embodiments where the camera captures an image of the scalp via a mirror.

FIG. 7A is a side view of an illumination device that is similar to the illumination device of FIG. 6 for embodiments where the mirror is deformable.

FIG. 7B is a top view of the illumination device of FIG. 7A.

FIG. 8 is a side view of an illumination device that is similar to the illumination device of FIGS. 1A to 1C for embodiments that further include a parting structure configured to create a part line of the user's hair during imaging.

FIG. 9 is a schematic view of a system for providing phototherapy treatments, capturing images, and/or characterizing images according to principles of the present disclosure.

FIG. 10 is a schematic representation of a process flow for the artificial intelligence (AI) classifier of the system of FIG. 9.

FIG. 11A is a top view illustration of a head of a patient with hair generally shown as shading along the scalp.

FIG. 11B is a top view illustration of the head of the patient from FIG. 11A overlaid with hair density mapping provided by the AI classifier of FIGS. 9 and 10.

FIG. 12 is a cross-sectional view of an illumination device that is similar to the illumination device of FIGS. 1A to 1C for embodiments that further include a scalp covering for holding down a user's hair during treatment.

FIG. 13 is a top view of a scalp covering with a mesh structure that may be implemented for the scalp covering of FIG. 12.

FIG. 14 is a top view of a scalp covering that includes a window that may be implemented for the scalp covering of FIG. 12.

FIG. 15 is a perspective view of the reflector of FIG. 12 for embodiments where the reflector forms a generally truncated cone structure with opposing openings.

FIG. 16 is a cross-sectional view of an exemplary structure of the reflector of FIGS. 12 and 15.

FIG. 17 is a cross-sectional view of another exemplary structure of the reflector of FIGS. 12 and 15.

FIG. 18 is a cross-sectional view of an illumination device that is similar to the illumination device of FIG. 12 for embodiments that include multiple cameras positioned to capture different views of a user's scalp.

### DETAILED DESCRIPTION

The embodiments set forth below represent the necessary information to enable those skilled in the art to practice the embodiments and illustrate the best mode of practicing the embodiments. Upon reading the following description in light of the accompanying drawing figures, those skilled in the art will understand the concepts of the disclosure and will recognize applications of these concepts not particularly addressed herein. It should be understood that these concepts and applications fall within the scope of the disclosure and the accompanying claims.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of the present disclosure. As used herein, the term “and/or” includes any and all combinations of one or more of the associated listed items.

It will be understood that when an element such as a layer, region, or substrate is referred to as being “on” or extending “onto” another element, it can be directly on or extend directly onto the other element or intervening elements may also be present. In contrast, when an element is referred to as being “directly on” or extending “directly onto” another element, there are no intervening elements present. Likewise, it will be understood that when an element such as a layer, region, or substrate is referred to as being “over” or extending “over” another element, it can be directly over or extend directly over the other element or intervening elements may also be present. In contrast, when an element is referred to as being “directly over” or extending “directly over” another element, there are no intervening elements present. It will also be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present.

Relative terms such as “below” or “above” or “upper” or “lower” or “horizontal” or “vertical” may be used herein to describe a relationship of one element, layer, or region to another element, layer, or region as illustrated in the Figures. It will be understood that these terms and those discussed above are intended to encompass different orientations of the device in addition to the orientation depicted in the Figures.

The terminology used herein is for the purpose of describing particular embodiments only and is not intended to be limiting of the disclosure. As used herein, the singular forms “a,” “an,” and “the” are intended to include the plural forms as well, unless the context clearly indicates otherwise. It will be further understood that the terms “comprises,” “comprising,” “includes,” and/or “including” when used herein specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this disclosure belongs. It will be further understood that terms used herein should be interpreted as having a meaning that is consistent with their meaning in the context of this specification and the relevant art and will not be interpreted in an idealized or overly formal sense unless expressly so defined herein.

Embodiments are described herein with reference to schematic illustrations of embodiments of the disclosure. As such, the actual dimensions of the layers and elements can be different, and variations from the shapes of the illustrations as a result, for example, of manufacturing techniques and/or tolerances, are expected. For example, a region illustrated or described as square or rectangular can have rounded or curved features, and regions shown as straight lines may have some irregularity. Thus, the regions illustrated in the figures are schematic and their shapes are not intended to illustrate the precise shape of a region of a device and are not intended to limit the scope of the disclosure. Additionally, sizes of structures or regions may be exaggerated relative to other structures or regions for illustrative purposes and, thus, are provided to illustrate the general structures of the present subject matter and may or may not be drawn to scale. Common elements between figures may be shown herein with common element numbers and may not be subsequently re-described.

Aspects of the present disclosure relate to wearable devices for delivering light energy to a scalp of a patient and/or capturing data from the scalp of the patient. Data capturing, for example imaging of the scalp, and particularly the hair follicles, is important in the phototherapeutic treatment of alopecia for various reasons, including identifying baseline scalp conditions and monitoring effectiveness of treatments over time. Additionally, data capturing of the scalp may be useful for characterizing and/or monitoring one or more of hair thickness, hair density, follicle density, dandruff, and/or a graying severity score (GSS). According to principles of the present disclosure, data capturing capabilities are integrated within illumination devices that deliver phototherapeutic treatments to the scalp. In this manner, repeatable and consistent data may be captured with each light treatment. This provides the ability to monitor the delivery of energy concurrently with treatments, and monitor and/or adjust parameters of light and/or dosing. Data capturing capabilities may include imaging capabilities where one or more integrated cameras within an illumination device are capable of capturing images of one or more regions of the scalp. In certain embodiments, data capturing capabilities include the ability to map larger regions of the scalp or even the entire scalp. As used herein, the terms illumination device, phototherapy device, and phototherapeutic device may be used interchangeably.

Before delving into specific details of various aspects of the present disclosure, an overview of various elements that may be included in exemplary phototherapeutic illumination devices for the scalp is provided for context. In certain aspects, illumination devices embody a wearable cap for positioning on a head of a patient during treatment. The illumination device includes one or more light-emitting elements for delivering light emissions to the scalp and one or more cameras for capturing images of the scalp.

Various types of light-emitting devices may be used for delivering light energy to the scalp of a patient. In certain embodiments, emissions may consist of non-coherent light, such as emissions generated by one or more light-emitting diodes (LEDs). According to principles of the present disclosure, arrangements of LEDs may provide improved scalp coverage with deeper penetration than coherent light sources. LEDs are arranged to emit overlapping cones of light that evenly cover the scalp for increased coverage of hair follicles. Coherent light sources, such as lasers, emit tightly focused beams that only cover discrete spots on the scalp. In certain embodiments, emissions of an illumination device may include combinations of coherent light and non-coherent light. Additionally, various aspects of the present disclosure, including the imaging capabilities described herein, are equally applicable to illumination devices with only coherent light sources.

In certain embodiments, multiple light-emitting devices of different peak wavelengths (e.g., having peak wavelengths differing by at least about 10 nanometers (nm), at least about 20 nm, at least about 30 nm, at least about 50 nm, at least about 75 nm, at least about 100 nm, or another threshold specified herein) may be provided. In certain embodiments, light of different peak wavelengths may be generated by different light-emitting devices contained in a single emitter package, wherein close spacing between adjacent emitters, such as LED chips, may provide integral color mixing. In certain embodiments, one or more arrays of light-emitting devices may be provided. For example, a first array of light-emitting devices may be configured to provide light of a first peak wavelength, and a second array of light-emitting devices may be configured to provide light of second peak wavelength. In certain embodiments, an array of multi-emitter packages may be provided, wherein emitters within a single package may provide the same or different peak wavelengths. In certain embodiments, an array of solid state emitter packages may embody packages further including second, third, fourth, and/or fifth solid state emitters, such that a single array of solid state emitter packages may embody two, three, four, or five arrays of solid state emitters, wherein each array is arranged to generate emissions with a different peak wavelength.

In certain embodiments, an illumination device for delivering light energy to a scalp of a patient may include one or more light-emitting devices devoid of a wavelength conversion material. In other embodiments, one or more light-emitting devices may be arranged to stimulate a wavelength conversion material, such as a phosphor material, a fluorescent dye material, a quantum dot material, and a fluorophore material.

In certain embodiments, one or more light-emitting devices may be arranged to provide substantially monochromatic light. In certain embodiments, one or more light-emitting devices may include a spectral output having a full width at half maximum value of less than 25 nm (or less than 20 nm, or less than 15 nm, or in a range of from 5 nm to 25 nm, or in a range of from 10 nm to 25 nm, or in a range of from 15 nm to 25 nm). In certain embodiments, one or more light-emitting devices may be arranged to provide emissions having a peak wavelength in a range of from 400 nm to 900 nm, or in a range of from 500 nm to 900 nm, or in a range of from 500 nm to 800 nm, or in a range of from 600 nm to 700 nm, or in a range of from 620 nm to 670 nm.

In certain embodiments, at least one light-emitting device may be arranged to provide emissions having a peak wavelength in a range of from 615 nm to 645 nm (or from 615 nm to 635 nm), and at least one light-emitting device may be arranged to provide emissions having a peak wavelength in a range of from 645 nm to 670 nm (or from 650 nm to 670 nm). In certain embodiments, at least one first light-emitting device may be arranged to provide emissions having a peak wavelength of about 620 nm, and at least one second light-emitting device may be arranged to provide emissions having a peak wavelength of about 660 nm. Such combination of wavelengths and wavelength ranges may be useful to provide anti-inflammatory effects, to promote vasodilation, and/or to reduce or block dihydrotestosterone (DHT). Anti-inflammatory effects may be useful to promote wound healing, to reduce acne blemishes, to promote facial aesthetics, and/or to treat atopic dermatitis and other topical dermatological disorders. Vasodilation may also be beneficial to treat androgenic alopecia or other topical dermatological disorders.

While certain aspects of the present disclosure include light-emitting devices with wavelengths for treating alopecia, various aspects are also applicable to illumination devices for treating other conditions alone or in combination with alopecia. For example, illumination devices as described herein may include at least one light-emitting device configured to produce light in a wavelength range and flux that improves wound healing, reduces acne blemishes, and/or alters the presence, concentration, or growth of pathogens, bacteria, and/or other microbes in or on living mammalian tissue receiving the light. In certain embodiments, exemplary peak wavelength ranges include one or more combinations of peak wavelengths in a range from 400 nm to 1000 nm, or 400 nm to 450 nm, or 410 nm to 430 nm, or 600 nm to 1000 nm, or 615 nm to 645 nm, and/or 645 nm to 670 nm.

In certain embodiments, any suitable combination of peak wavelengths disclosed herein may be used in combination for desired therapeutic effects (e.g., vasodilation, inflammation reduction, nitric oxide generation, nitric oxide release, and antimicrobial functions). In certain embodiments, a combination of wavelengths may be provided during the same time window, during overlapping but non-coincident time windows, or during non-overlapping time windows.

In certain embodiments, at least one first light emitter and at least one second light emitter (which may be embodied in a first array of light emitters and a second array of light emitters) may be arranged to provide different peak wavelengths selected from one of the following combinations (a) to (f): (a) the first peak wavelength is in a range of from 620 nm to 640 nm (or from 615 nm to 635 nm) and the second peak wavelength is in a range of from 650 nm to 670 nm; (b) the first peak wavelength is in a range of from 520 nm to 540 nm and the second peak wavelength is in a range of from 650 nm to 670 nm; (c) the first peak wavelength is in a range of from 400 nm to 420 nm (or from 410 nm to 430 nm) and the second peak wavelength is in a range of from 620 nm to 640 nm; (d) the first peak wavelength is in a range of from 400 nm to 420 nm (or from 410 nm to 430 nm) and the second peak wavelength is in a range of from 650 nm to 670 nm; (e) the first peak wavelength is in a range of from 400 nm to 420 nm (or from 410 nm to 430 nm) and the second peak wavelength is in a range of from 495 nm to 515 nm; and (f) the first peak wavelength is in a range of from 400 nm to 420 nm (or from 410 nm to 430 nm) and the second peak wavelength is in a range of from 520 nm to 540 nm.

In addition to various sources of light, the principles of the present disclosure are also applicable to one or more other types of directed energy sources. As used herein, a directed energy source may include any of the various light sources previously described, and/or an energy source capable of providing one or more of heat, infrared (IR) heating, resistance heating, radio waves, microwaves, soundwaves, ultrasound waves, electromagnetic interference, and electromagnetic radiation that may be directed to a target body tissue. Combinations of visual and non-visual electromagnetic radiation may include peak wavelengths in a range from 180 nm to 4,000 nm. Illumination devices as disclosed herein may include a light source and another directed energy source capable of providing directed energy beyond visible light. In other embodiments, the other directed energy source capable of providing directed energy beyond visible light may be provided separately from illumination devices of the present disclosure.

In certain embodiments, one or more light-emitting devices may provide a fluence of at least 1 joule per square centimeter (J/cm²), at least 3 (J/cm²), or at least 5 (J/cm²) when energized to emit light. In certain embodiments, one or more light-emitting devices may provide a radiant flux in a range of from 5 milliwatts per square centimeter (mW/cm²) to 60 mW/cm². In certain embodiments, one or more light-emitting devices may be arranged to provide substantially steady state light. In certain embodiments, one or more light-emitting devices may be arranged to provide multiple discrete pulses of light.

In certain embodiments, light-emitting devices may be arranged on one or more flexible substrates configured to conform to the shape of a wearable cap for positioning on the head of a patient. The flexible substrate may comprise a flexible printed circuit board (FPCB) supporting at least one light-emitting device. In certain embodiments, a FPCB may include a polyimide-containing layer and at least one layer of copper or another electrically conductive material. In certain embodiments, a light-transmissive layer (e.g., an encapsulant or lens) may be arranged to cover and/or arranged in contact with at least a portion of a FPCB and any light emitter(s) supported thereon. An exemplary material for the light-transmissive layer is silicone, which may be applied by any suitable means such as molding, dipping, spraying, dispensing, printing, or the like. In certain embodiments, substantially all surfaces (e.g., front and back surfaces) of a FPCB may be covered with encapsulant material. In certain embodiments, the total thickness of an encapsulated flexible LED including embedded light emitters may be in a range of 1 millimeter (mm) to 5 mm, or in a range of from 1 mm to 3 mm, not including standoffs. In certain embodiments, the FPCB comprises a flexible polymer film, polyester (PET), polyimide (PI), polyethylene naphthalate (PEN), polyetherimide (PEI), fluoropolymers (FEP), copolymers, etc.

In certain embodiments, at least one standoff is configured to be arranged between the FPCB and the scalp of the patient, with the at least one standoff including a standoff height that exceeds a height of emitters supported by the FPCB. Preferably, the at least one standoff comprises a light-transmissive material such as silicone, PET, polyethylene terephthalate glycol (PET-G), etc. Various steps of forming an encapsulated FPCB with standoffs may include defining electrical traces on the FPCB; mounting, forming or otherwise affixing one or more light-emitting elements on the FPCB; forming standoffs or standoff portions; and encapsulating various structures including the light-emitting elements, the FPCB, and optionally encapsulating standoffs or standoff portions. The order of the preceding steps may be altered, and in certain embodiments, portions or the entirety of at least some standoffs may be devoid of encapsulant.

In certain embodiments, standoffs or standoff portions may be molded, placed, formed, printed, adhered, or otherwise applied to a face of a FPCB prior to encapsulation, and the standoffs or standoff portions may thereafter be partially or fully encapsulated together with one or more light-emitting elements and one or more portions of the FPCB. In other embodiments, standoffs or standoff portions may be placed, formed, printed, adhered, or otherwise applied to a face of a FPCB after the FPCB and light-emitting elements have been encapsulated. In various embodiments, standoffs or standoff portions may be formed concurrently with an encapsulation process for the light transmissive layer, such as by molding, printing, spraying, or other deposition methods.

Standoffs or standoff portions may be formed by cross-linkable materials selectively applied or formed along regions of a FPCB, and such materials may be activated by appropriate means (e.g., heat, photonic energy, chemical activation, or the like) before, during, or after an encapsulation step.

In certain embodiments, standoff height, standoff shape, light-emitting element spacing, and light element optical distribution may be selected to permit adjacent light-emitting elements to provide an overlapping beam pattern on a scalp of a patient. In certain embodiments, an array of multiple standoffs may be formed on, in, or over an encapsulant material. In certain embodiments, each standoff within an array has substantially the same size, shape, and/or durometer. In other embodiments, different standoffs within an array may include different sizes, shapes, and/or durometers. In certain embodiments, one or more standoffs may include suitable shapes and/or materials to provide light-focusing utility, light-diffusing utility, and/or light-scattering utility. In certain embodiments, one or more standoffs may include one or more wavelength conversion materials (e.g., phosphors, quantum dots, fluorophores, or the like) and provide wavelength conversion utility. In certain embodiments, one or more standoffs may include suitable shapes and/or materials to provide light reflection utility. In certain embodiments, one or more standoffs may be placed apart from one or more light-emitting elements. In other embodiments, one or more standoffs may be intentionally placed on or over one or more light-emitting elements, with the standoff(s) serving to transmit, shape, and/or otherwise affect light received from one or more light-emitting elements.

Illumination devices for delivering light energy to a scalp of a patient may include a FPCB with multiple interconnected panels and a plurality of bending regions defined in and between the multiple panels to allow the FPCB to provide a concave shape to cover at least a portion of a cranial vertex of a patient. In certain embodiments, the FPCB is formed with a shape that includes various extensions and/or flaps that conform to a concave shape with sharp bending regions. In certain embodiments, openings are provided between portions of adjacent panels to permit transport of heat and fluids (e.g., perspiration). In certain embodiments, a fabric covering may be arranged to cover the FPCB, with the fabric covering preferably being breathable to permit transport of heat and fluid transport (e.g., evaporation of sweat). In certain embodiments, the fabric covering may include an adjustable closure arranged to permit an opening circumference of the fabric covering to be adjusted. If the FPCB is contained within the fabric covering, then adjustment of the closure may selectively compress a portion of the FPCB and therefore also permit an opening circumference of the FPCB to be adjusted. In certain embodiments, the FPCB and the fabric covering are arranged to accommodate outward expansion and inward contraction to permit standoffs of the FPCB to contact the scalp of the patient.

In certain embodiments, the FPCB may form a plurality of curved panels projecting generally outwardly and downwardly from a central frame to substantially conform to portions of the cranium. Gaps may be provided between portions of curved panels to accommodate outward expansion and inward contraction, and to enable dissipation of heat generated by the at least one light-emitting device associated with the FPCB.

In certain embodiments, a flexible shaping member having a generally concave interior may be arranged to receive a FPCB. The flexible shaping member may be provided between the FPCB and fabric covering to accommodate outward expansion and inward contraction to permit the plurality of standoffs to contact the scalp of the patient. In certain embodiments, a flexible shaping member may be fabricated from a suitable polymeric material.

In certain embodiments, an illumination device may include an electronics housing. The electronics housing may include driver circuitry (or at least a portion of driver circuitry) configured to energize at least one light-emitting device for impingement of light on the scalp of a patient. In certain embodiments, the electronics housing may include one or more of a user interface, sensory interface, charging interface, data interface, signal input, signal output, and/or display elements. In certain embodiments, an energy storage device (e.g., a battery) may be retained by a battery holder pivotally (or otherwise movably) coupled to the electronics housing. Such movable coupling may permit relative movement between the battery holder and electronics housing to permit the phototherapy device to accommodate a variety of patients having different head sizes and shapes.

In certain embodiments, operation of an illumination device as disclosed herein may be responsive to one or more signals generated by one or more sensors or other elements. Various types of sensors are contemplated, including temperature sensors, photosensors, image sensors, proximity sensors, pressure sensors, chemical sensors, biosensors, accelerometers, moisture sensors, oximeters, current sensors, voltage sensors, and the like. Other elements that may affect impingement of light and/or operation of a device as disclosed herein include a timer, a cycle counter, a manually operated control element, a wireless transmitter and/or receiver (as may be embodied in a transceiver), a laptop or tablet computer, a mobile phone, or another portable digital device. Wired and/or wireless communication between a device as disclosed herein and one or more signal generating or signal receiving elements may be provided.

According to principles of the present disclosure, illumination devices may include one or more data capture devices capable of capturing data of the scalp and corresponding hair follicles. In certain aspects, data capture devices comprise cameras capable of capturing images of the scalp and corresponding hair follicles. In other aspects, data capture devices may include video capturing devices, light-based topography sensors such as Lidar devices, and three-dimensional scanning devices, among others. Such data and/or images may be useful for characterizing and/or monitoring one or more of hair thickness, hair density, follicle density, dandruff, and/or a graying severity score (GSS). The one or more data capture devices may be incorporated within the illumination device, such as on or near the FCBP. In other embodiments, the one or more data capture devices may reside in other locations, such as along a covering of the illumination device and/or proximate the light-transmissive layer. In the context of cameras, one or more lenses may also be arranged to enhance magnification of captured images. In certain embodiments, the one or more cameras and corresponding lenses are capable of capturing microscopic images with 100× to 200× magnification or more. By way of example, a 10× or 20× lens may be combined with a camera with 10× zoom capabilities. Exemplary cameras include micro cameras with a size or largest dimension of 1.5 mm or less, or 1 mm or less. Other exemplary cameras include camera modules with cameras and lenses integrated on a larger board, clip-on microscopic cameras and lenses, and/or other medical use cameras, such as those present in otoscopes or endoscopes. Cameras may embody one or more of charged-coupled devices (CCD) and/or complementary metal oxide semiconductors (CMOS) cameras, among others.

In certain embodiments, an illumination device as disclosed herein may be configured to prevent unauthorized usage beyond an authorized number of treatment cycles. For example, a number of treatment cycles of the device may be incremented and stored in a counter or other memory element. When the number of treatment cycles reaches a predetermined limit, operation of the illumination device may be reversibly or irreversibly disabled. In certain embodiments, when the number of treatment cycles reaches a predetermined limit, a signal may be communicated to a user to notify the user that a predetermined limit of a number of treatment cycles has been reached, and a user may be prompted to either (i) purchase a new device or component thereof, or (ii) purchase the ability to continue using the device for a specified number of additional cycles or for a specified additional time period. In certain embodiments, one or more signals relating to cycle usage and/or enabling a user to purchase additional usage may be communicated via wired or wireless means. In certain embodiments, a user may download an application for use on a personal computer, a tablet computer, a mobile phone, or another portable digital device, and the application may provide cycle usage information and/or permit the user to purchase additional cycles or purchase additional usage time to continue using the device.

In certain embodiments, upon detection of a specified number of uses of the device, a light-emitting device may be configured to produce a disabling signal adapted to irreversibly disable the device to prevent further operation of the device. In certain embodiments, the disabling signal may include at least one of a voltage spike and a current spike arranged to damage at least one circuit element. In certain embodiments, a light-emitting device includes a power supply circuit arranged to provide at least one conditioned power signal for use by a microcontroller of the device and/or the at least one light-emitting device, and a disabling signal may be adapted to irreversibly disable at least one element of the power supply circuit. In certain embodiments, at least one fusible link may be arranged in electrical communication with the at least one light-emitting device, and a disabling signal may be adapted to open the at least one fusible link to prevent current from being supplied to the at least one light-emitting device. In certain embodiments, at least one fusible link may be arranged in electrical communication with at least one light emitter and/or a light emitter driver circuit. In certain embodiments, impingement of light on living tissue and/or

operation of an illumination device as disclosed herein may be responsive to one or more temperature signals. For example, a temperature condition may be sensed on or proximate to a FPCB; at least one signal indicative of the temperature condition may be generated; and operation of a device for delivering light energy to a scalp of a patient may be controlled responsive to the at least one signal. Such control may include initiation of operation, deviation (or alteration) of operation, or termination of operation of light-emitting elements. In certain embodiments, thermal foldback protection may be provided at a threshold temperature (e.g., >42° Celsius) to prevent a user from experiencing burns or discomfort. In certain embodiments, thermal foldback protection may trigger a light-emitting device to terminate operation, reduce current, or change an operating state in response to receipt of a signal indicating an excess temperature condition.

In certain embodiments, a proximity sensor may be arranged proximate to a portion of a FPCB to determine when a FPCB is proximate to a surface (e.g., scalp) to be illuminated and used for safety of a patient by reducing flux when not proximate to the surface. Additionally, one or more proximity sensors may be used to initiate data capturing of integrated data capture devices such as cameras. In one example, the proximity sensor may initiate a progression of data and/or images as the illumination device is placed on the scalp of a user.

In certain embodiments, a device for delivering light energy to a scalp of a patient may include a user-perceptible visible signaling element (e.g., one or more lights, a LED display, an alphanumeric display, mobile app, or the like) arranged to generate a visible signal and/or a user-perceptible audible signaling element (e.g., a speaker, a buzzer, an alarm generator, or the like) arranged to generate an audible signal. In certain embodiments, at least one of the visible signal and the audible signal is indicative of operating status or charging status of the device. In certain embodiments, at least one of the visible signal and the audible signal is indicative of count of operating cycles of the device.

In certain embodiments, a device for delivering light energy to a scalp of a patient as disclosed herein may include a memory element to store information indicative of one or more sensor signals and/or captured data and/or images. Such information may be used for detecting device usage, assessing patient status, assessing patient improvement, and assessing function of the device. In certain embodiments, information indicative of one or more sensor signals and/or captured data and/or images may be transmitted via wired or wireless means (e.g., via Bluetooth, WiFi, Zigbee, or another suitable protocol) to a mobile phone, a computer, a data logging device, or another suitable device that may optionally be connected to a local network, a wide-area network, a telephonic network, or other communication network. In certain embodiments, a data port (e.g., micro USB or other type) may be provided to permit extraction or interrogation of information contained in a memory.

The exemplary embodiments described below are provided in the context of one or more cameras for capturing one or more images before, during, and after phototherapy. In addition to cameras and corresponding imaging, the principles disclosed are applicable to any data capture device, such as video capturing devices, light-based topography sensors such as Lidar devices, and three-dimensional scanning devices, among others. In this manner, one or more other types of data capture devices may be substituted in place of any of the cameras described below for the exemplary figures. Captured data and/or images may be useful for characterizing and/or monitoring one or more of hair thickness, hair density, follicle density, dandruff, and/or a graying severity score (GSS).

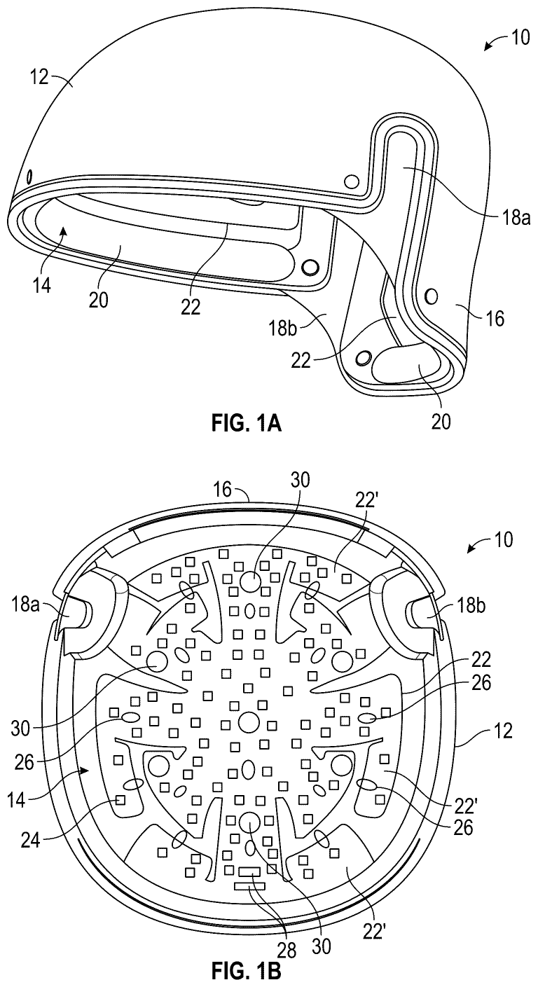

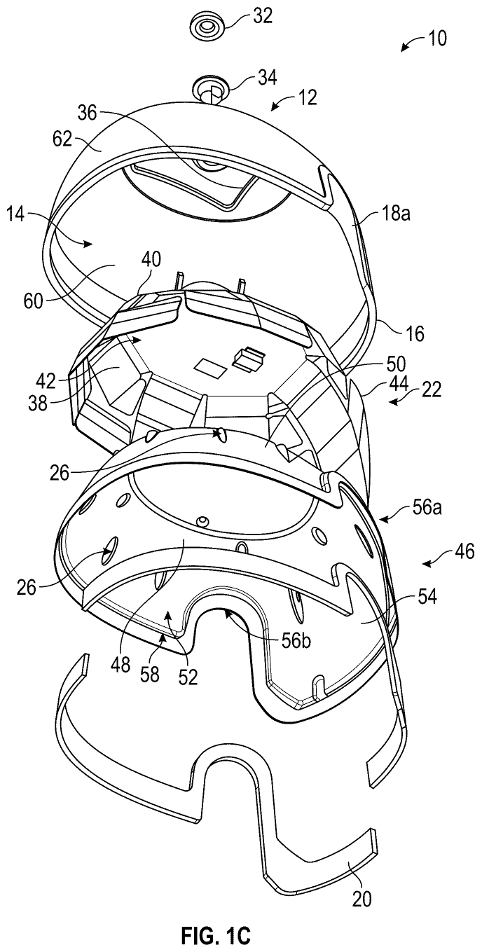

FIG. 1A is a perspective view of an illumination device 10 embodying a wearable cap for delivering light to the scalp of a patient. The illumination device 10 includes a cap 12 formed of one or more fabrics or materials, such as cotton, open or closed cell polyethylene foam, polyester, and rayon, among others. The illumination device 10 may be formed in a variety of different shapes and sizes depending on the head dimensions of the patient. In certain embodiments, the cap 12 is a flexible cap with a stretchable material for accommodating flexibility of the illumination device 10 for a variety of head sizes. The illumination device 10 forms a concavity 14 generally sized and shaped to receive an upper portion of a head of a patient. In certain embodiments, the illumination device 10 includes a posterior extension 16 configured to cover the lower rear portion of a patient's head (e.g., the nape, posterior hairline, occipital protuberance, and/or proximate thereto, etc.). Proximate to the posterior extension 16 and towards a center of the illumination device 10, a flex arc (e.g., left flex arc 18a and right flex arc 18b) is positioned to allow the posterior extension 16 to more easily flex outward to accommodate varying head sizes. The flex arcs 18a and 18b may embody a stretchable material that resides within a cutout portion of the illumination device 10 to accommodate outward and/or inward flexibility. In certain embodiments, a padding 20 may be provided within the concavity 14 for comfort. From the perspective of FIG. 1A, a flexible substrate 22, such as a FPCB, is visible within the concavity 14 and along the posterior extension 16 facing the concavity 14.

FIG. 1B is a bottom view of the illumination device 10 of FIG. 1A illustrating a view within the concavity 14. As illustrated, the flexible substrate 22 is formed with a shape that includes various interconnected flaps 22′ that promote flexibility for conforming to the concavity 14. An array of light-emitting devices 24, such as LEDs, are provided across the flexible substrate 22 in positions to provide broad coverage of light emissions. In FIG. 1B, a number of standoffs 26 are positioned in various locations across the flexible substrate 22 to provide separation between the flexible substrate 22 and the scalp of a patient. In certain embodiments, one or more sensors 28 may also be located on one or more portions of the flexible substrate 22. The sensors 28 may embody one or more combinations of sensors as described above, including but not limited to temperature sensors, photosensors, image sensors, proximity sensors, and the like. By way of example, the sensors 28 In FIG. 1B are located near a front of the illumination device 10 proximate a forehead position of the patient when in use. Alternatively, the sensors 28 may be located on any other portion of the flexible substrate 22, depending on the application.

As further illustrated in FIG. 1B, one or more cameras 30 may be integrated within the illumination device 10 for capturing images of the scalp before, during, and/or after phototherapy treatments. By way of example, seven cameras 30 are illustrated in FIG. 1B in locations across the flexible substrate 22. As described above, the cameras 30 may embody miniature cameras with sizes adept for positioning in multiple locations. In this regard, one or more portions or even an entire scalp may be imaged and mapped by the illumination device 10. In other embodiments, any number of cameras 30 may be present, including a single camera depending on the application. For illustrative purposes, the relative sizing of the light-emitting devices 24 and the cameras 30 may not necessarily be to scale. In FIG. 1B, the light-emitting devices 24 are generally smaller than the cameras 30. However, in other embodiments, an individual camera 30 may occupy less surface area than an individual light-emitting device 24.

FIG. 1C is an exploded view of the illumination device 10 of FIGS. 1A and 1B. In certain embodiments, the cap 12 may include a seal plug 32 removably attached to and covering an electronic connection port 34 at a top of the cap 12. The electronic connection port 34 is mechanically attached to an electronics receptacle 36 arranged at a top of the cap 12. The seal plug 32 covers and protects the electronic connection port 34 from damage when not in use. The electronics receptacle 36 may be configured to receive an electronics subassembly of the flexible substrate 22. For example, the electronics receptacle 36 may be mechanically attached and secured to the electronics subassembly to prevent relative movement therebetween. The electronic connection port 34 (with the electronics receptacle 36) may provide mechanical and/or electronic connectivity between the illumination device 10 and an electronic device (e.g., computer, smartphone, etc.) external to the illumination device 10. The electronic connection port 34 may also embody an electronic connector (e.g., power cord, USB cord, etc.) to receive electrical power and/or electronic data (e.g., operational parameters). In certain embodiments, wireless communication may be provided between the illumination device 10 and an external electronic device. In certain embodiments, a battery operatively coupled to the flexible substrate 22 may be inductively charged (e.g., wirelessly charged).

The flexible substrate 22 includes a proximal surface 38 (which may variously be referred to as a bottom surface, lower surface, inner surface, inside surface, or surface proximate to the patient) and a distal surface 40 (which may variously be referred to as a top surface, upper surface, outer surface, outside surface, or surface further from the patient). The flexible substrate 22 forms a concavity 42 generally sized and shaped to receive at least an upper portion of the head of a patient and may include a posterior extension 44 configured to cover the lower rear portion of a patient's head (e.g., the nape, posterior hairline, occipital protuberance, and/or proximate thereto, etc.). The proximal surface 38 may include one or more of the light-emitting devices, at least one camera, and one or more sensors as described above for FIG. 1B.

A light-transmissive layer 46, such as a flexible lens, includes a proximal surface 48 (e.g., inner surface, inside surface, surface proximate to the patient) and a distal surface 50 (e.g., outer surface, outside surface, surface further from the patient). The light-transmissive layer 46 forms a concavity 52 generally sized and shaped to the head of a patient, and may include a posterior extension 54 configured to cover the lower part of a back of a patient's head (e.g., the nape, posterior hairline, occipital protuberance, and/or proximate thereto, etc.). Proximate the posterior extension 54 (but towards the center of the light-transmissive layer 46), the light-transmissive layer 46 may include flex arcs 56a, 56b, which allow the posterior extension 54 to more easily flex outward to accommodate varying head sizes. The light-transmissive layer 46 may be molded and may have a thickness of approximately 0.02 inches (in). to 0.06 in. (e.g., approximately 0.033 in.). In certain embodiments, the light-transmissive layer 46 includes a plurality of lenses with a lens density in a range of from about 10 to about 80 lenses per inch (LPI), or from about 20 to about 60 LPI, or from about 30 to about 50 LPI, or about 40 LPI, although other dimensions may be used. In certain embodiments, the light-transmissive layer 46 may have a lenticular surface for directing light therethrough. Depending on the materials used, if the light-transmissive layer 46 has a thickness smaller than about 0.02 in., then undesirable wrinkling or crinkling may result, and if the light-transmissive layer 46 has a thickness greater than 0.06 in., it may be insufficiently flexible or stretchable to accommodate head sizes of different patients. As described herein, the light-transmissive layer 46 may embody an encapsulant or an encapsulant material as described above.

In certain embodiments, a light-transmissive layer 46 may include a padding recess 58 along a peripheral edge of the light-transmissive layer 46 to receive the foam padding 20 (such that the foam padding 20 contacts the proximal surface 48). In this manner, the padding recess 58 and the foam padding 20 may be generally complementary to each other in size and/or shape. In certain embodiments, the padding recess 58 and foam padding 20 may be configured to extend along the entire peripheral edge of the light-trans missive layer 46 or a portion thereof. The foam padding 20 provides an additional layer of comfort and a compressible layer for an improved fit to the patient's head. In certain embodiments, the foam padding 20 may be removably attached (e.g., by Velcro), such that the foam padding 20 may be washable and/or replaceable.

The flexible substrate 22 is positioned between the cap 12 and the light-transmissive layer 46. More specifically, the flexible substrate 22 is positioned within the cap concavity 14 such that the flexible substrate distal surface 40 is proximate to a proximal surface 60 of the cap 12, opposite a distal surface 62 of the cap 12. The light-transmissive layer 46 is positioned within the concavity 42 of the flexible substrate 22 such that the distal surface 50 of the light-transmissive layer 46 is positioned proximate the proximal surface 38 of the flexible substrate 22. In this manner, when the flexible cap 12, flexible substrate 22, and light-transmissive layer 46 are assembled together, the concavities and peripheral edges thereof are generally aligned with one another. In the same manner, the cap concavity 14, flexible substrate concavity 42, and light-transmissive layer concavity 52 are all generally aligned with one another; the cap posterior extension 16, the flexible substrate posterior extension 44, and the light-transmissive layer posterior extension 54 are all generally aligned with one another; and the cap flex arcs 18a, 18b and light-transmissive layer flex arcs 56a, 56b are generally aligned with one another.

The plurality of standoffs 26 may include top standoffs positioned at or along a top of the light-transmissive layer 46 and side standoffs positioned at or along a side of the light-transmissive layer 46. The standoffs 26 are positioned between the light-transmissive layer 46 and the flexible substrate 22. In certain embodiments, the standoffs 26 may be attached to (e.g., by adhesive) or may be integrally formed with the light-transmissive layer 46 (e.g., by molding the standoffs 26 concurrently with the light-transmissive layer 46). More specifically, for each standoff 26, a standoff proximal end contacts and extends from the light-transmissive layer distal surface 50, and a standoff distal end may contact the flexible substrate proximal surface 38. The height of the standoffs 26 may be greater than a height of the light-emitting devices to prevent the light-transmissive layer 46 from contacting the light-emitting devices. In this manner, the standoffs 26 maintain a minimum distance between the light-transmissive layer 46 and the flexible substrate 22.

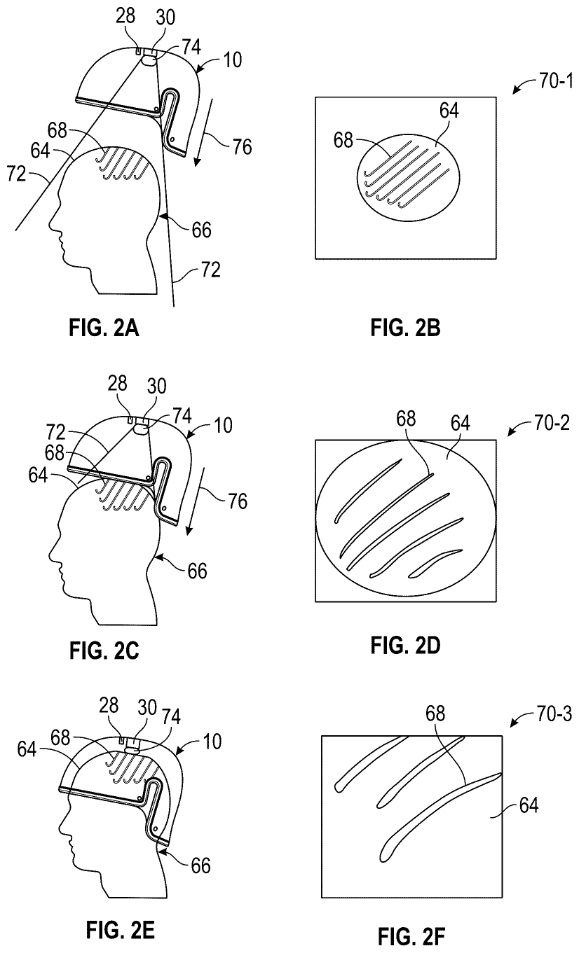

FIGS. 2A to 2F illustrate capturing progressive images from various distances as the illumination device 10 of FIGS. 1A to 1C is positioned on a scalp 64 of a user 66. In certain embodiments, the camera 30 may capture a series of images for characterization of the scalp 64, which may include a view of the entire scalp 64 along with one or more magnified images of a portion of the scalp 64. As will be described later in greater detail, the series of images may be used to quantify an amount of hair 68 on the scalp 64 and/or an average amount of the hair 68 for an area of the scalp 64.

FIG. 2A is a view of the illumination device 10 of FIGS. 1A to 1C at an initial step for positioning the illumination device 10 on the scalp 64 of the user 66. FIG. 2B is a representation of a first image 70-1 of the scalp 64 taken when the illumination device is positioned according to FIG. 2A. As illustrated in FIG. 2A, the illumination device 10 is positioned above the user 66 in a position so that a field of view 72 of the camera 30 encompasses the entire scalp 64. FIG. 2C is a view of the illumination device 10 at a subsequent step to FIG. 2A where the illumination device 10 is positioned closer to the scalp 64, but not in a position to begin light treatment. FIG. 2D is a representation of a second image 70-2 of the scalp 64 taken when the illumination device is positioned according to FIG. 2C. Finally, FIG. 2E is a view of the illumination device 10 at a subsequent step to FIG. 2C where the illumination device 10 is positioned proximate the scalp 64 and in a position to begin light treatment. FIG. 2F is a representation of a third image 70-3 of the scalp 64 taken when the illumination device 10 is positioned according to FIG. 2E. In this manner, the illumination device 10 is capable of capturing images 70-1 to 70-3 of the scalp 64 at various distances and/or magnifications. While a progression of the three images 70-1 to 70-3 is illustrated, the principles are applicable to any number of captured images, including continuous image capturing as the illumination device 10 is positioned for light treatment. In certain embodiments, the camera 30 may continue to capture images during and after light treatment. In still further embodiments, the camera 30 may capture a progression of images as the illumination device 10 is being removed in reverse order from FIGS. 2F to 2A.

In certain embodiments, the illumination device 10 includes the sensor 28 embodied as a proximity sensor. The camera 30 may be configured to initiate and/or terminate image capturing based on signals detecting the presence or absence of the scalp 64 by the proximity sensor 28. In certain embodiments, the camera 30 comprises an optic 74 configured to focus on the scalp 64 through the progression of images 70-1 to 70-3. The optic 74 may embody an auto-focusing lens that adjusts to various distances between the camera 30 and the scalp 64 as the illumination device 10 is moved in a direction 76 toward the user 66. In other embodiments, the optic 74 is a flexible optic that is configured to change its shape when in contact with the scalp 64. For example, in FIGS. 2A and 2C, the optic 74 forms a generally outwardly curved surface relative to the camera 30 to provide a larger field of view 72. When positioned on the scalp 64 as illustrated in FIG. 2E, the optic 74 compresses to a generally flat shape along the scalp 64, thereby narrowing the field of view 72 from FIGS. 2A and 2C.

In certain embodiments, the illumination device 10 may be configured such that the camera 30 and/or optic 74 initiate capturing of images on contact with the scalp 64 as illustrated in FIG. 2E. Images may continue to be captured while the illumination device 10 is in contact with the scalp 64 during light treatment, and imaging may be stopped when the illumination device 10 is removed from and not in contact with the scalp 64.

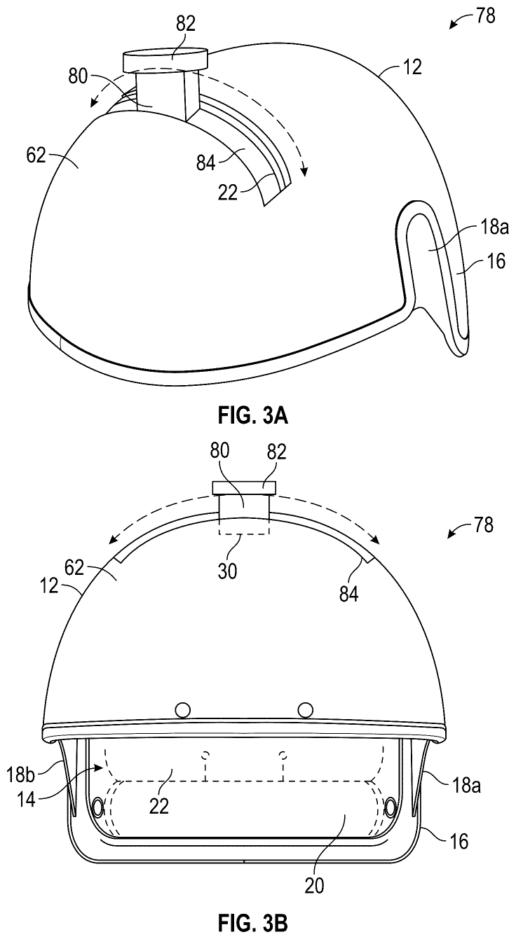

FIG. 3A is a top perspective view of an illumination device 78 that is similar to the illumination device 10 of FIGS. 1A to 1C for embodiments where a position of the camera 30 is adjustable. FIG. 3B is a front view of the illumination device 78 of FIG. 3A. As illustrated, the illumination device 78 includes a camera adjustment structure 80 configured to adjust a position of the camera 30 relative to the cap 12. In this regard, the position of the camera 30 may be moved to capture images from an increased area of the user's scalp while the illumination device 78 is worn by the user. Accordingly, a position of the camera 30 is adjustable relative to a position of light-emitting devices (e.g., 24 of FIG. 1B). For illustrative purposes, the position of the camera 30 below the cap 12 is illustrated in dashed lines. In certain embodiments, the camera adjustment structure 80 may be mechanically connected to the camera 30 and capable of being moved manually by a user and/or another person assisting with treatment. In other embodiments, movement of the camera 30 may be controlled by the driver circuitry of the illumination device 78 in an automated manner as part of a treatment protocol. A focus adjustment structure 82 may also be included that provides focus control for the camera 30, either manually by the user or automated by the illumination device 78. The focus adjustment structure 82 may be accessible from the distal surface 62 of the cap 12. In certain embodiments, the focus adjustment structure 82 comprises a rotatable knob on the camera adjustment structure 80.

By way of example, the camera 30 of FIGS. 3A and 3B is adjustable along a transverse or side-to-side direction of the illumination device 78 as illustrated by the superimposed dashed-line arrows. In certain embodiments, a movement channel 84 or track for the camera 30 may be formed through one or more of the cap 12, the flexible substrate 22, and the light-transmissive layer (i.e., 46 of FIG. 1C). For manual adjustment, a user may control a position of the camera 30 via moving the camera adjustment structure 80 along the movement channel 84. As indicated above, in automated mode, the movement is controlled by the illumination device 78. While the example of FIGS. 3A and 3B illustrate transverse movement, the principles described are equally applicable to other movement directions alone or in combination with the transverse direction, including a front-to-back direction.

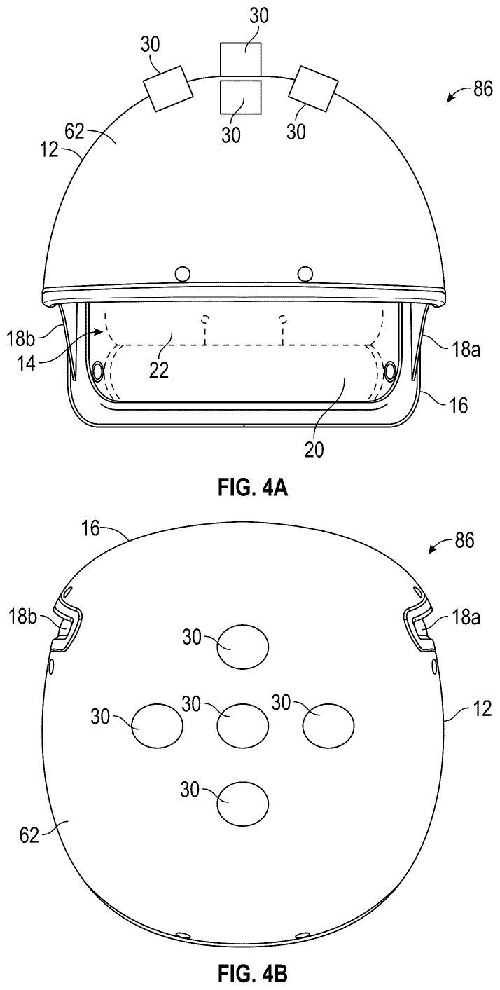

FIG. 4A is a front view of an illumination device 86 that is similar to the illumination device 78 of FIGS. 3A and 3B for embodiments that include an array of cameras 30. FIG. 4B is a front view of the illumination device 86 of FIG. 4A. As illustrated, the cameras 30 form an array or grid across the illumination device 86. In this manner, the illumination device 86 is capable of concurrently capturing multiple images from different areas of a user's scalp. While four cameras 30 are illustrated, the principles are applicable to any number of cameras 30 across the illumination device 86. With the array of cameras 30, the illumination device 86 is similar to the illumination device of FIGS. 1A to 1C. In this regard, the illumination device 86 may capture multiple images of different areas of the scalp for each of the positions illustrated in FIGS. 2A, 2C, and 2E.

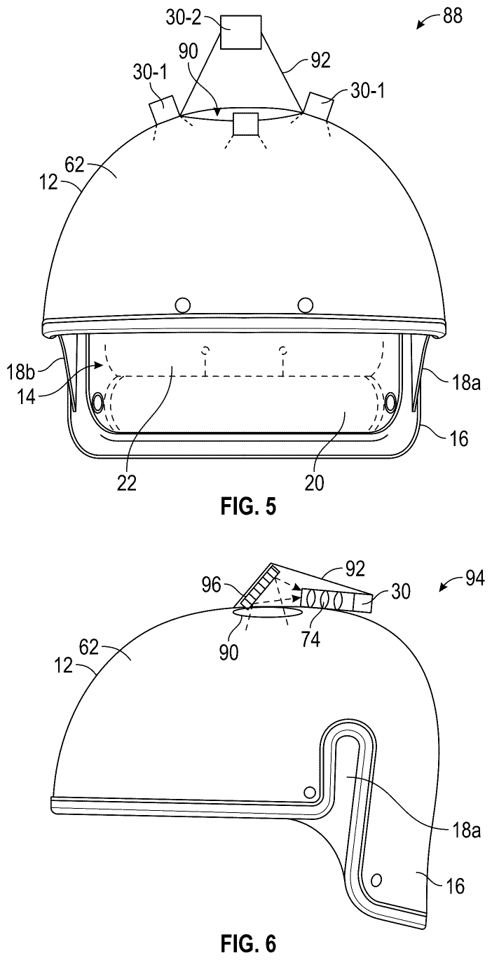

FIG. 5 is a front view of an illumination device 88 that is similar to the illumination device 86 of FIGS. 4A and 4B for embodiments that include different cameras 30-1, 30-2 that capture different fields of view. For example, one or more first cameras 30-1 may be positioned to capture one or more near field images when the illumination device 88 is positioned on a user as illustrated in FIG. 2E. In such a position for use, one or more second cameras 30-2 may be positioned to capture one or more far field images of the scalp. In certain embodiments, the second camera 30-2 may capture far field images through an opening 90 of the of the illumination device 88 that exposes the scalp. In this manner, the illumination device 88 is able to concurrently capture near field and far field images of a user's scalp while the illumination device 88 is in position for use. In FIG. 5, the second camera 30-2 is generally illustrated above the one or more first cameras 30-1 for far field imaging. In certain embodiments, the second camera 30-2 may be fixed in such position by way of a housing 92 or mechanical support structure that extends from the cap 12. The housing 92 may embody a separate piece or an integral single-piece extension of another portion of the illumination device 88.

FIG. 6 is a side view of an illumination device 94 that is similar to the illumination device 86 of FIGS. 4A and 4B for embodiments where the camera 30 captures an image of the scalp via a mirror 96. As illustrated, the camera 30 may be positioned on the distal surface 62 of the cap 12 to image the scalp through the opening 90 via reflection from the mirror 96. In certain embodiments, the camera 30 and one or more optics 74 associated with the camera 30 are oriented in a direction towards the mirror 96 along the distal surface 62. The camera 30, optics 74, and mirror 96 may reside within the housing 92 for mechanical support relative to the opening 90. In this manner, the camera 30 is configured to capture a fixed field of view through the opening 90 during use. In certain embodiments, the arrangement of the mirror 96 provides the ability for the camera 30 to have a low-profile.

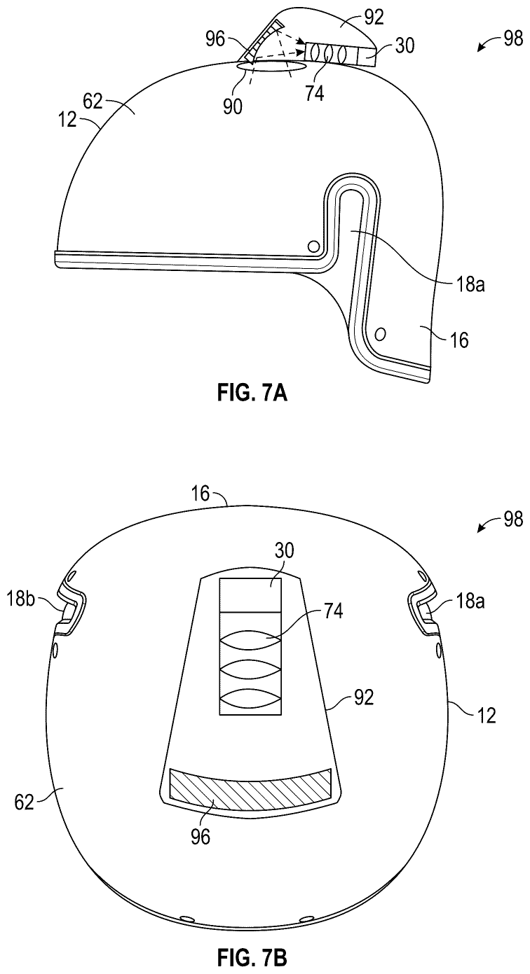

FIG. 7A is a side view of an illumination device 98 that is similar to the illumination device 94 of FIG. 6 for embodiments where the mirror 96 is deformable. FIG. 7B is a top view of the illumination device 98 of FIG. 7A. In certain embodiments, the mirror 96 is configured to flex forward and/or backward, such as when the illumination device 98 is fitted to a user's head. The compression of the mirror 96 may form a curved reflective surface for imaging via the camera 30. As illustrated, the curved reflective surface may be concave relative to the camera 30 in certain compressed positions. In this manner, the camera 30 may image a more focused area of the scalp with a smaller field of view compared with the illumination device 94 of FIG. 6.

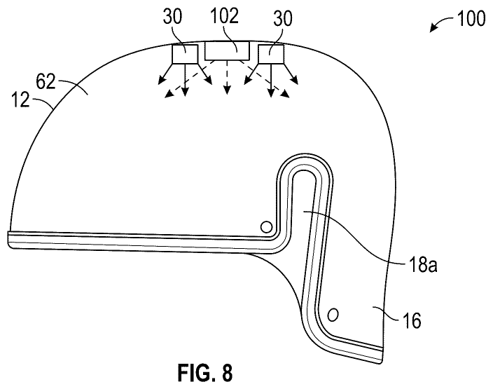

FIG. 8 is a side view of an illumination device 100 that is similar to the illumination device 10 of FIGS. 1A to 1C for embodiments that further include a parting structure 102 configured to create a part line of the user's hair during imaging. In this manner, as the illumination device 100 is placed on a user's scalp, the parting structure 102 may move hair to create the part line for imaging. Such an arrangement may be useful for ensuring images include portions of the scalp along with hair follicles. In certain embodiments, the parting structure 102 embodies a fan configured to direct sufficient air on a portion of the user's scalp to create the part line. In other embodiments, the parting structure 102 may embody a structure that mechanically contacts and moves the hair, such as a comb feature, that is implemented alone or in combination with a fan. The principles described for the parting structure 102 are applicable for implementation in any of the previous embodiments, including FIGS. 1A to 7.

While the previously described embodiments for FIGS. 1A to 7 describe imaging in combination with phototherapy, the principles described are also applicable to embodiments that omit illumination of the scalp. Accordingly, any of the previously described embodiments may omit the light-emitting devices or be operated to capture images without electrically activating the light-emitting devices. For embodiments that do not include light-emitting devices, illumination devices as described herein may be referred to as image capturing devices, such as an image capturing cap. The ability to capture images of the scalp in a repeatable and uniform manner may be useful for characterizing and/or diagnosing various conditions of hair loss and/or other conditions of the scalp. In certain embodiments, such imaging may be useful for characterizing and/or monitoring progress of hair transplants, with or without phototherapy. For embodiments that include phototherapy from light-emitting devices, the imaging described herein provides the ability to tailor light characteristics before, during, and after treatment. For example, the light-emitting devices may illuminate the scalp with generally white light for capturing images, followed by various wavelengths of light targeting detected hair loss and/or other conditions of the scalp. In still further embodiments, one or more cameras as previously described may be configured to filter certain wavelengths for band imaging, such as short wavelength blue light, infrared imaging, etc. In certain embodiments, one or more cameras as previously described may be configured for hyperspectral imaging of the scalp to characterize one or more of melanin and blood oxygen levels, among others.

According to principles of the present disclosure, illumination devices as disclosed herein are well suited for capturing images of a user's scalp and providing such images to an external device for further analysis. In certain embodiments, captured images are characterized via artificial intelligence (AI) to identify, count, map, and/or characterize hair, hair follicles, and other conditions of the scalp. The AI may embody an AI classifier, such as a deep neural network trained to recognize features of hair and the scalp in images based on an image library of previously stored or accesses images. The AI classifier may identify what an image is, what is in it, and count how many of something (e.g., hair, hair follicles) is in the image. In this manner, the AI classifier may provide classification of captured image data into various categories. In certain embodiments, the AI classifier provides feature-detection in the ability to identify and create various subcategories, or parameters, of the features of the whole image data set it is trained on. Accordingly, the AI classifier may identify what is in the captured image and the corresponding probability. For example, the identification may include one or more of hair, hair thickness, hair density, hair color, and/or follicle count. The AI classifier may be trained by previously loaded and curated data, as well as continued learning through accumulation of captured image data over time. In certain embodiments, a suitably trained AI classifier with ongoing patient tracking may be adept at learning best practices and/or medications for implementation.

In certain embodiments, external devices and/or systems associated with AI classifier capabilities may broadly represent any type or form of computing device or system capable of executing computer-readable instructions, such as those contained within the modules described herein. In their most basic configuration, these computing device(s) may each include at least one memory device and at least one physical processor.

In some examples, the term “memory device” generally refers to any type or form of volatile or non-volatile storage device or medium capable of storing data and/or computer-readable instructions. In one example, a memory device may store, load, and/or maintain one or more of the modules described herein. Examples of memory devices include, without limitation, random access memory (RAM), read only memory (ROM), flash memory, hard disk drives (HDDs), solid-state drives (SSDs), optical disk drives, caches, variations or combinations of one or more of the same, or any other suitable storage memory. In some examples, the term “physical processor” generally refers to any type or form of hardware-implemented processing unit capable of interpreting and/or executing computer-readable instructions. In one example, a physical processor may access and/or modify one or more modules stored in the above-described memory device. Examples of physical processors include, without limitation, microprocessors, microcontrollers, central processing units (CPUs), field-programmable gate arrays (FPGAs) that implement softcore processors, application-specific integrated circuits (ASICs), portions of one or more of the same, variations or combinations of one or more of the same, or any other suitable physical processor.

Although various modules may be provided as separate elements, the modules described and/or illustrated herein may represent portions of a single module or application. In addition, in certain embodiments one or more of these modules may represent one or more software applications or programs that, when executed by a computing device, may cause the computing device to perform one or more tasks. For example, one or more of the modules described and/or illustrated herein may represent modules stored and configured to run on one or more of the computing devices or systems described and/or illustrated herein. One or more of these modules may also represent all or portions of one or more special-purpose computers configured to perform one or more tasks. In addition, one or more of the modules described herein may transform data, physical devices, and/or representations of physical devices from one form to another. For example, one or more of the modules recited herein may receive sensor or image data to be transformed, transform the data, output a result of the transformation to control impingement of light onto living tissue such as the scalp, use the result of the transformation to control impingement of nitric-oxide modulating light onto the living tissue, and/or store the result of the transformation to control impingement of nitric-oxide modulating light onto the living tissue. Additionally or alternatively, one or more of the modules recited herein may transform a processor, volatile memory, non-volatile memory, and/or any other portion of a physical computing device from one form to another by executing on the computing device, storing data on the computing device, and/or otherwise interacting with the computing device.

In certain embodiments, the term “computer-readable medium” generally refers to any form of device, carrier, or medium capable of storing or carrying computer-readable instructions. Examples of computer-readable media include, without limitation, transmission-type media, such as carrier waves, and non-transitory-type media, such as magnetic-storage media (e.g., hard disk drives, tape drives, and floppy disks), optical-storage media (e.g., compact disks (CDs), digital video disks (DVDs), and BLU-RAY disks), electronic-storage media (e.g., solid-state drives and flash media), and other distribution systems.

Illumination devices as described herein may be incorporated as part of a larger system that provides characterization, control and/or management of treatments. In this regard, the illumination devices as described herein may represent any form factor including illumination devices that are at least partially controlled or managed by all or a portion of a larger system. The system may be configured to provide control and/or management for phototherapy treatments provided by one or more of the illumination devices based on one or more characteristics of the scalp that are provided. The one or more characteristics may be classified from captured images of the scalp and/or other characteristics that may be measured. A server and a server-side application may further provide control and/or management for illumination devices based on data collected from multiple other illumination devices.

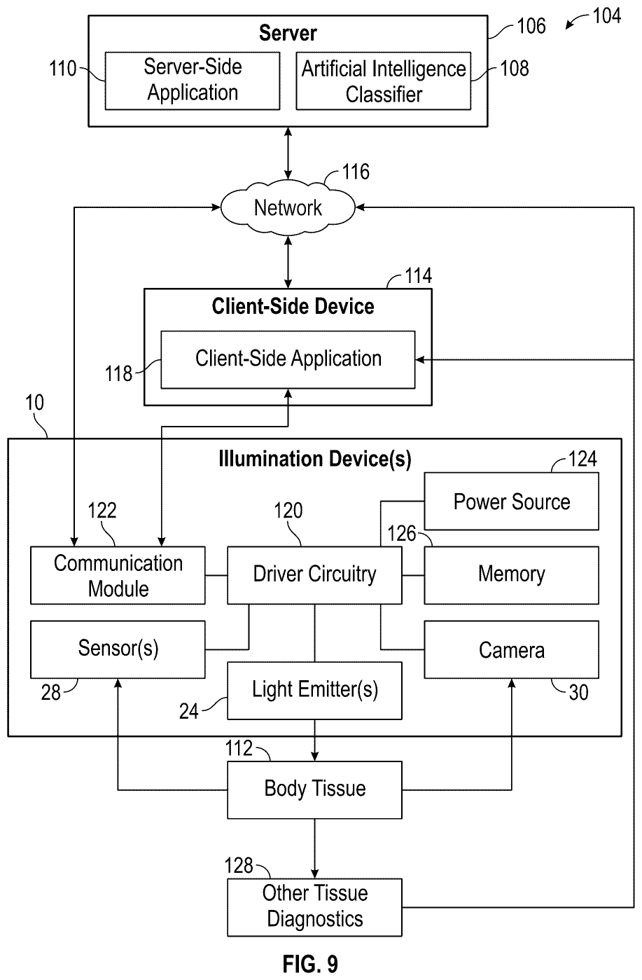

FIG. 9 is a schematic view of a system 104 for providing phototherapy treatments, capturing images, and/or characterizing images. In FIG. 9, the illumination device 10 is generally illustrated in schematic view and may refer to any of the illumination devices previously described for FIGS. 1A to 7. The system 104 may include a server 106 with an AI classifier 108, or AI library, that is populated with suitable data, including but not limited to clinical trial data and data (e.g., images and other sensor data) captured by other illumination devices in practice. A server-side application 110 may receive image data specific to a targeted body tissue 112 (e.g., a scalp), compare the image data previously accessed by the AI classifier 108, and formulate a tailored phototherapy treatment for the body tissue 112. In certain embodiments, the server-side application 110 is integrated as a portion of the AI classifier 108. Accessible data for the AI classifier 108 may be continually updated and refined based on populated data to continuously improve the ability to characterize images of the scalp and/or provide tailored phototherapy treatments with increased efficacy. As used herein, accessible data for the AI classifier 108 may refer to a collection of data (e.g., images and/or sensor data) that correspond to previously identified characteristics of hair, hair follicles, dandruff, GSS, and other scalp features.

In certain embodiments, the server 106 is in communication with a client-side device 114 via a network 116. The client-side device 114 may include a client-side application 118 for managing, controlling, and/or communicating with the illumination device 10. In at least one embodiment, the client-side application 118 may be configured to collect (e.g., as part of a clinical trial) image and/or sensor data from illumination devices and/or user feedback. The server 106 and client-side device 114 generally represent any type or form of computing device capable of reading computer-executable instructions. Examples of the server 106 and the client-side device 114 include, without limitation, laptops, tablets, desktops, servers, cellular phones, personal digital assistants (P DAs), multimedia players, embedded systems, wearable devices (e.g., smart watches, smart glasses, etc.), routers, switches, gaming consoles, combinations of one or more of the same, or any other suitable computing device. In at least one example, the client-side device 114 may represent a user's computing device to which the user has paired the illumination device 10.

The network 116 generally represents any medium or architecture capable of facilitating communication or data transfer. Examples of the network 116 include, without limitation, an intranet, a wide area network (WAN), a local area network (LAN), a personal area network (PAN), the Internet, power line communications (PLC), a cellular network (e.g., a global system for mobile communications (GSM) network), or the like. The network 116 may facilitate communication or data transfer using wireless or wired connections. In one embodiment, the network 116 may facilitate communication between the server 106 and either the client-side device 114 or the illumination device 10.

In this manner, the AI classifier 108 may be utilized by one or more of the server-side application 110 and the client-side application 118 to recognize patterns in image data and/or sensor data collected from the targeted body tissue 112 to infer one or more characteristic and/or conditions of the body tissue 112. Accordingly, the system 104 may be configured to provide tailored phototherapy treatments that may be administered by the illumination device 10 in response to the inferred conditions. Additionally, the system 104 may be configured for mapping hair and scalp conditions for monitoring progress of treatments.

As described herein, the system 104 may provide an exemplary embodiment for a method that includes accessing data related to the body tissue 112, generating at least one parameter based on the data related to the body tissue 112, and sending the parameter to the illumination device 10 for irradiating the body tissue 112 based on the at least one parameter. Such a method may be implemented with any system and/or device configuration beyond the exemplary embodiment provided by the system 104 of FIG. 9.

The illumination device 10 may include the one or more light-emitting devices 24, one or more cameras 30, one or more sensors 28, and corresponding driver circuitry 120. Captured image data and sensor data may be relayed back to the server 106 for analysis. The illumination device 10 may further include a communication module 122 that facilitates communication with the client-side device 114 and the client-side application 118. Alternatively, the communication module 122 may be configured to communicate directly with the network 116 and the server 106 without the client-side device 114. The communication module 122 may provide such communication via any number of manners, including Bluetooth, wired and/or wireless internet connections, a cellular network, analog communication such as one or more pre-programmed buttons of the illumination device 10, or any other form of analog or digital communication.

The illumination device 10 may include a power source 124 that includes any type of internal power source and/or connections to an external power source. For example, the power source 124 may embody a portable power source and/or energy storage device that is provided within the illumination device 10, such as a replaceable battery and/or a rechargeable battery. For rechargeable embodiments, the illumination device 10 may include a port (e.g., 34 of FIG. 1C that embodies a universal serial bus port, a power plug, or the like) for providing a connection to an external power source or another device, such as the client-side device 114, for recharging. In certain embodiments, the port may also facilitate data transfer and communication via the communication module 122. The power source 124 may be configured for direct connections to an external power source with or without recharging capabilities, including a wired and/or a plug-direct configuration to the external power source. As used herein, the external power source may include a hardwired electrical connection such as a wall plug or any type of wired or portable external energy storage device. In still further embodiments, the external power source coupled to the power source 124 of the illumination device 10 may embody a human factor power source at the client-side that provides power responsive to human movements, such as walking and/or chewing by a user. The external power source may further embody renewable energy sources, including solar and/or wind sources, that provide power to and or recharging of the power source 124. In certain applications, the system 104 may include a solar element or panel that may be worn by a user of the illumination device 10, such as solar hat, a solar sleeve, or any other form of solar clothing.

The illumination device 10 may include a memory device 126 that stores various drive algorithms and/or control schemes for the driver circuitry 120 based on data received from the server 106. The memory device 126 may further be configured to store data and diagnostic information collected at the targeted body tissue 112 for communication with the server 106. As described above, the memory device 126 may include any type or form of a volatile and/or a non-volatile storage device or any medium capable of storing data and/or computer-readable instructions. For example, the memory device 126 may include, without limitation, RAM, ROM, flash memory, HDDs, SSDs, optical disk drives, caches, and variations or combinations of one or more of the same, or any other suitable storage memory.

While the driver circuitry 120, the communication module 122, and the memory device 126 are illustrated as separate blocks or elements, each of the driver circuitry 120, the communication module 122, and the memory device 126 may also represent elements within a combined overall control circuitry module for the illumination device 10.

As described above, the illumination device 10 may be configured to capture images and/or other diagnostic information of the body tissue 112 (e.g., the scalp) for analysis via any number of the cameras 30 and sensors 28. Captured images may include one or more visible-light images, infrared images, ultraviolet images, hyperspectral images, images measuring light within a predetermined range of wavelengths, images measuring light within two or more different predetermined ranges of wavelengths, reflected resonance images, reflected wave images, and ultrasound images. The sensors 28 may include one or more of temperature sensors, photo sensors, image sensors, proximity sensors, blood pressure or other pressure sensors, chemical sensors, biosensors (e.g., heart rate sensors, body temperature sensors, sensors that detect presence or concentration of chemical or biological species, or other conditions), accelerometers, moisture sensors, oximeters, such as pulse oximeters, current sensors, voltage sensors, and the like. The cameras 30 and sensors 28 may work together as needed to perform various functions. The camera 30 may further provide precise location information for the body tissue 112 based on camera pixelated measurements, and global positioning system (GPS) data, among others.

In combination data captured by the illumination device 10, the system 104 may also be configured to receive other tissue diagnostics 128 that are collected separately from the illumination device 10. The other tissue diagnostics 128 may include external cameras and sensors that are similar to any of the above-described embodiments of the sensor 28 and the camera 30. Additionally, the other tissue diagnostics 128 may be collected by any number of other medical imaging devices. In further embodiments, the other tissue diagnostics 128 may include information provided by a user and/or a medical professional based on a physical examination and/or diagnostic tests administered to the body tissue 112 and the corresponding user.

The captured images and/or sensor data from the illumination device 10 and/or provided by the other tissue diagnostics 128 may be relayed to one or more of the client-side device 114 and/or server 106 via the network 116 for analysis. Accordingly, the captured images and/or sensor data may be compared with large volumes of images of known scalp conditions and corresponding sensor data accessible by the AI classifier 108. In this regard, the system 104 may determine characteristics of the body tissue 112 including but not limited to identification and/or mapping of hair, hair thickness, hair density, hair follicles, dandruff, GSS, and other scalp conditions. The accessible data may initially be populated with as many images as possible that are then added to with each subsequent new patient data. This provides the system 104 with the ability to expand and evolve for improved identification so that appropriate and up-to-date treatments may be delivered to the body tissue 112. The system 104 may further provide functionality that includes determining corresponding treatment costs to provide real-time billing, appropriate insurance claims, and exchange of payments. In certain embodiments, the system 104 may further be used to monitor the body tissue 112 and recommend a subsequent anti-inflammatory treatment or other medications for use.

In this manner, patient outcomes may continually be optimized by the system 104 based on collective information received by multiple ones of the illumination devices 10 across a large volume of users. Optimization may refer to a best-available or a continually-improved outcome such as one or more of prevention, treatment, cure, and follow-up treatments for one or more conditions that may be present. The server 106 may further identify other recommended treatments for the body tissue 112 that may be implemented in combination with the illumination device 10, such as one or more medications that may be administered to further improve or optimize patient outcomes.

The system 104 may provide various treatment algorithms that may include any number of changeable attributes for the illumination device 10, such as one or more peak wavelengths, radiant fluxes, irradiances, exposure times, and corresponding light doses that may be provided to the body tissue 112. Doses may be provided by a series of energy sources or alternatives of the same energy source (e.g., different peak wavelengths of light) that may be deployed in a singular or multiple fashion according to any of the previously described embodiments. As described herein, treatments and/or doses may be provided with appropriate safety, efficacy and time per treatment for achieving the improved patient outcomes.

According to further implementations, any of the above-described elements and functions of the system 104 may be provided with less automated configurations. For example, a more simplified version of the system 104 may include a configuration where a user may click-through a menu or simply press pre-configured buttons on the illumination device 10 and/or the client-side device 114 to select a particular treatment program. In another example, a user may progress through one or more steps on the illumination device 10 and/or the client-side device 114 to provide images or other diagnostic information via the illumination device 10. In certain embodiments, one or more of the client-side device 114 and the illumination device 10 may also include a local AI classifier so that treatment algorithms may be provided without having to communicate with the external server 106. In such embodiments, the local AI classifier may be periodically synchronized with the AI classifier 108 of the server 106 according to routine intervals.

In certain embodiments, the AI classifier 108 is configured to take rotational line samples across an image and measure bumps and a hair count. Count estimates for hair may be based on one or more sample points of a distribution. Hair counts provided by the AI classifier 108 may be manually verified to ensure accuracy. In certain embodiments, the AI classifier 108 is configured to determine if an image feature is hair or hair follicles by analysis of shape profiles. The AI classifier 108 may further identify hair ends using contrast differences on a cluster of pixels from capture images. In certain embodiments, contrast may be enhanced with a melanin filter.

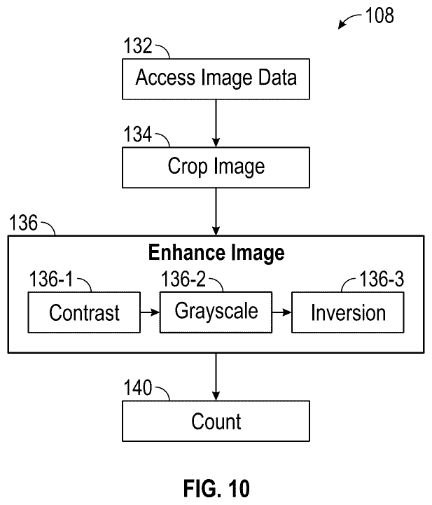

FIG. 10 is a schematic representation of a process flow for the AI classifier 108 of FIG. 9. At a first step 132, image data is imported or otherwise accessed. The image data may refer to one or more images captured by the camera 30 of the illumination device 10 of FIG. 9. At a second step 134, the image may be cropped to an area that includes a target area of the scalp. In certain embodiments, the AI classifier 108 is configured to crop a larger area of the scalp based on identification of hair in the target area. A scale of the image may be derived from meta data, assumed based on camera specific information, or estimated based on width of hairs in the image and/or a head size for far field images of the user. In a third step 136, the image is enhanced for a subsequent counting step by one or more of adjusting contrast 136-1, converting to grayscale 136-2, and image inversion 136-3. In certain embodiments, the image may be enhanced for counting progressing sequentially through each of contrast adjustment 136-1, grayscale conversion 136-2, and image inversion 136-3. In this manner, an increased contrast image of the target area is subject to grayscale conversion followed by image inversion so that hairs may appear white for counting against a dark background. In a fourth step 140, the enhanced image is then subject to counting by the AI classifier 108. In certain embodiments, counting comprises edge detection of the image to detect pixels with brightness changes as compared to surrounding pixels, filtering out noise, and creating a data file where pixels correspond with brightness changes based on set thresholds. The number of pixels along hair edges may then be counted to determine a hair density as compared with a total count of image pixels. In certain embodiments, the AI classifier 108 may then determine and/or filter out hair crossovers. In still further embodiments, multiple images from a scalp of a user may be characterized using the AI classifier 108 to provide hair density mapping.

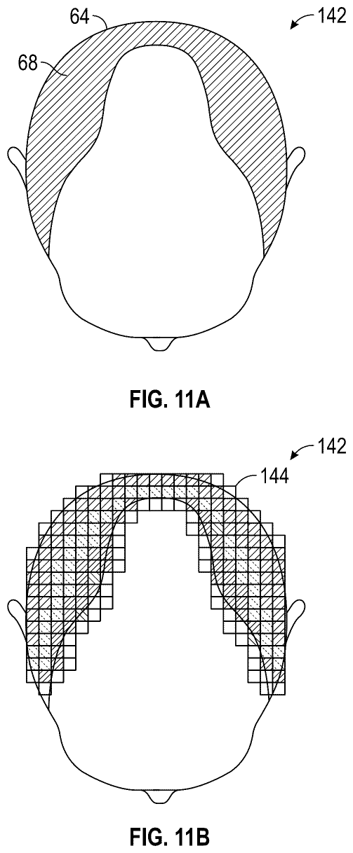

FIGS. 11A and 11B are top view illustrations representing an exemplary hair density map according to principles of the present disclosure. FIG. 11A is a top view illustration of a head 142 of a patient with hair 68 generally shown as shading along the scalp 64. FIG. 11B is a top view illustration of the head 142 of the patient from FIG. 11A overlaid with hair density mapping provided by the AI classifier 108 of FIGS. 9 and 10. In FIG. 11B, a series of squares 144 define image areas individually characterized by the AI classifier 108. In this regard, each square 144 defines an area of the scalp 64 that was imaged and characterized. The squares 144 may be stitched together according to location and overlaid over the illustration of the head 142. The hair density mapping adjusts the appearance of the squares 144, such as dark to light or different colors, based on relative hair density classified for each square 144. By way of example, each square 144 may represent a 1 centimeter (cm) by 1 cm area. In other embodiments, the resolution may be increased such that each square 144 represents an even smaller area of the head 142. With such capabilities, phototherapeutic treatments may be tailored based on hair density maps. For example, a hair density map may be collected for a user and one or more of peak wavelength, intensity, and dosing may be spatially adjusted by an illumination device based on the hair density map.

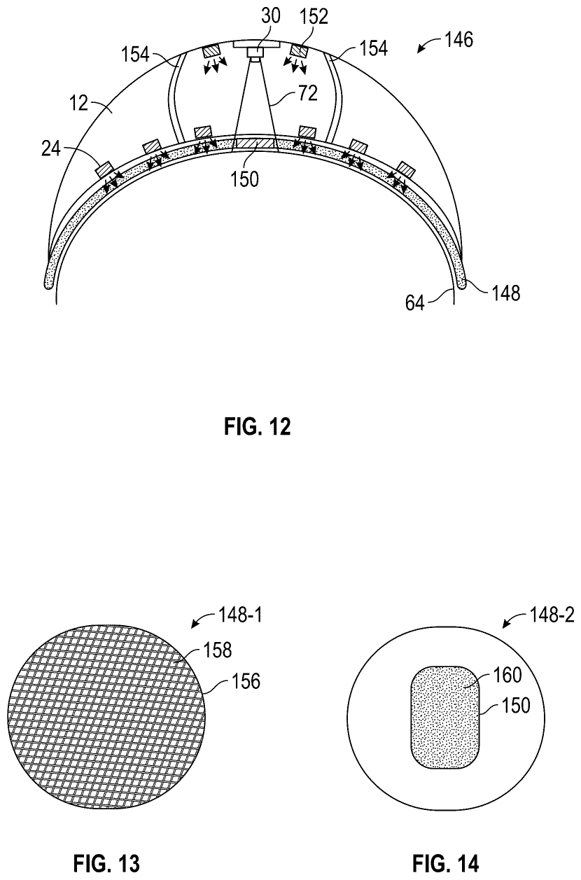

FIG. 12 is a cross-sectional view of an illumination device 146 that is similar to the illumination device 10 of FIGS. 1A to 1C for embodiments that further include a scalp covering 148 for holding down a user's hair during treatment. The scalp covering 148 may be formed of a material and/or a structure that permits light to pass therethrough. In various embodiments, the scalp covering 148 forms a structure that conforms to the scalp 64 during use, thereby applying suitable pressure to hold down or compress any hair of the user against the scalp 64. In this manner, light from the light-emitting devices 24 may more readily reach the scalp 64. Furthermore, the scalp covering 148 may provide enhanced visibility of the scalp 64 and/or hair in images captured by the camera 30. In certain embodiments, the scalp covering 148 may be integrated with the illumination device 146. For example, the scalp covering 148 may be secured to a portion of the illumination device 146, such as lower portions of the cap 12, in a position to conform to the scalp 64 during use. Alternatively, the scalp covering 148 may embody a separate structure that is first placed on the scalp 64 before the illumination device 146.

Depending on the structure of the scalp covering 148, a window 150 may be integrated within a portion of the scalp covering 148 proximate the field of view 72 of the camera 30. The window 150 may embody a transparent window. In further embodiments, the window 150 may be formed with one or more antireflective materials or films to further encourage light passage. In other embodiments, the structure of the scalp covering 148 may be such that the window 150 is not required. For example, the scalp covering 148 may be formed of a transparent material and/or the scalp covering 148 may embody a mesh material with numerous open portions. In certain embodiments, one or more additional light-emitting devices 152 may be provided that are arranged to provide general illumination to the scalp 64 during imaging. As illustrated, the additional light-emitting devices 152 may be positioned proximate the camera 30 and/or the window 150. The additional light-emitting devices 152 may be configured to provide generally white light and/or other specific wavelengths depending on the type of imaging. In still further embodiments, the illumination device 146 may include a reflector 154 positioned to reflect and/or collect light from the additional light-emitting devices 152 to enhance illumination of the portion of the scalp 64 during imaging.

FIGS. 13 and 14 illustrate exemplary structures for the scalp covering 148 of FIG. 12. FIG. 13 is a top view of a scalp covering 148-1 with a mesh structure 156 that may be implemented for the scalp covering 148 of FIG. 12. The mesh structure 156 may be formed of a material, such as nylon or an elastic material, the promotes conforming to a user's scalp with sufficient pressure to hold the hair in place. The mesh structure 156 forms numerous openings 158 to permit light therethrough and/or image capturing. FIG. 14 is a top view of a scalp covering 148-2 that includes the window 150 and may be implemented for the scalp covering 148 of FIG. 12. As described above for FIG. 12, the window 150 may be transparent and/or the window 150 may include an antireflective material 160, such as one or more antireflective films.

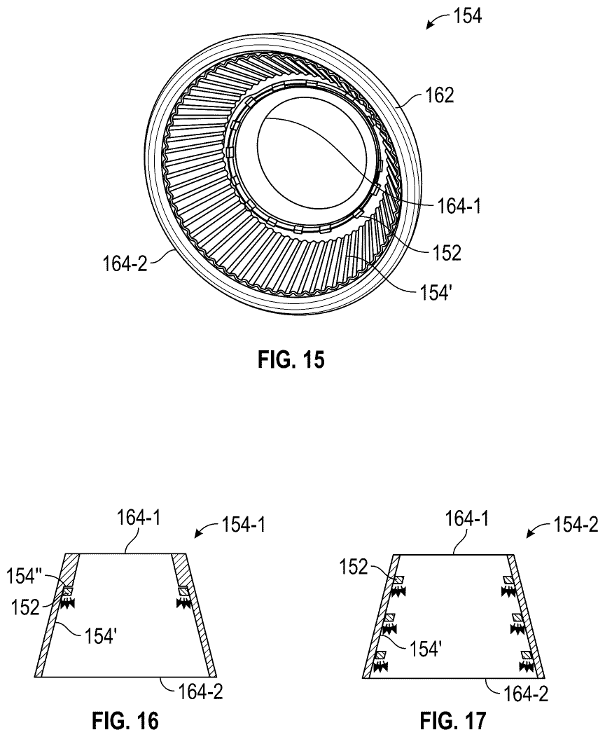

FIG. 15 is a perspective view of an exemplary structure of the reflector 154 of FIG. 12. In certain embodiments, the reflector 154 forms a truncated cone structure 162 with opposing first and second openings 164-1, 164-2. The first opening 164-1 may be smaller than the second opening 164-2. The first opening 164-1 is sized such that the camera 30 of FIG. 12 may be positioned proximate the first opening 164-1 in a forward-facing direction to capture images of the scalp. In this manner, the second opening 164-2 is configured to reside closer to the scalp during use. As illustrated, the additional light-emitting devices 152 may be provided along one or more inner sidewalls 154′ of the reflector 154 between the first and second openings 164-1, 164-2. In this manner, the reflector 154 may form a light chamber for collecting and redirecting light from the additional light-emitting devices 152 to illuminate the scalp during image capturing. In certain embodiments, the inner sidewall 154′ may include a structure, such as scalloping, to further scatter and/or redirect light toward the scalp.

FIGS. 16 and 17 illustrate exemplary structures for the reflector 154 of FIGS. 12 and/or 15. FIG. 16 is a cross-sectional view of a reflector 154-1 that may be implemented as the reflector 154 of FIGS. 12 and 15. As illustrated, the sidewall 154′ may form one or more ledges 154″ on which the additional light-emitting devices 152 reside. In this configuration, the additional light-emitting devices 152 may be oriented to provide light toward the second opening 164-2.

FIG. 17 is a cross-sectional view of another reflector 154-2 that may be implemented as the reflector 154 of FIGS. 12 and 15. Instead of the ledges 154″ of FIG. 16, the additional light-emitting devices 152 may have structures that preferentially emit light toward the second opening 164-2. In this regard, the additional light-emitting devices 152 may be mounted on planar portions of the sidewalls 154′.

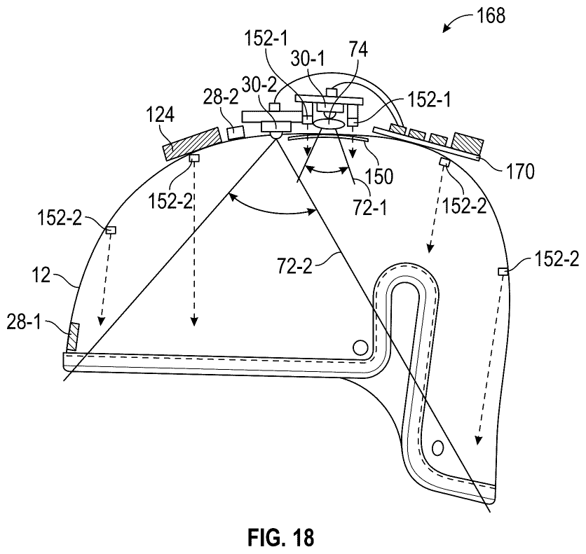

FIG. 18 is a cross-sectional view of an illumination device 168 that is similar to the illumination device 146 of FIG. 12 for embodiments that include multiple cameras 30-1, 30-2 positioned to capture different views of a user's scalp. For example, a first camera 30-1 may embody a camera similar to the camera 30 of FIG. 12. As illustrated, the first camera 30-1 is positioned to have a first field of view 72-1 that is focused for capturing a close-up image of a portion of a user's scalp. The first camera 30-1 may include the optic 74, such as a lens, as previously described and the first field of view 72-1 may be provided through the window 150 as described above for FIG. 12. For close-up imaging, the first camera 30-1 may be configured to capture images similar to the third image 70-3 of FIG. 2F. In certain embodiments, first additional light-emitting devices 152-1 are arranged proximate the first camera 30-1 for providing imaging light during image capturing. The imaging light may embody generally white light and/or other specific wavelengths depending on the type of imaging.

The illumination device 168 may further include a second camera 30-2 that is configured to image a larger portion of the user's scalp than the first camera 30-1. In certain embodiments, the second camera 30-2 may have a second field of view 72-2 that encompasses a majority of the user's scalp, such as the views illustrated in FIGS. 11A and 11B. In this regard, the camera 30-2 may be configured to capture images similar to the first image 70-1 of FIG. 2B. As illustrated, the second field of view 72-2 may overlap and/or encompass the first field of view 72-1. In certain embodiments, second additional light-emitting devices 152-2 are arranged for providing imaging light during image capturing with the second camera 30-2. As with the first additional light-emitting devices 152-1, imaging light from the second additional light-emitting devices 152-2 may embody generally white light and/or other specific wavelengths depending on the type of imaging. The second additional light-emitting devices 152-2 may be positioned across larger portions of the illumination device 168 than the first additional light-emitting devices 152-1 in order to provide imaging light across larger portions of the user's scalp that correspond with the second field of view 72-2.

As further illustrated in FIG. 18, the illumination device 168 may include multiple proximity sensors 28-1, 28-2 that are configured to detect the proximity of the scalp at different distances relative to the illumination device 168. For example, a first proximity sensor 28-1 may be positioned to detect when the user has positioned the illumination device 168 in place on the head. In this regard, the first proximity sensor 28-1 may be configured to initiate up-close imaging from the first camera 30-1. A second proximity sensor 28-2 may be positioned to detect when the user has positioned the illumination device 168 a certain distance in a spaced relationship over the scalp. In this regard, the second proximity sensor 28-2 may be configured to initiate distance imaging from the second camera 30-2.

In certain embodiments, a microprocessor 170 may be integrated with the illumination device 168 for controlling operation of the cameras 30-1, 30-2. The microprocessor 170 may control operation of the cameras 30-1, 30-2 based on signals received from the proximity sensors 28-1, 28-2. In certain embodiments, the microprocessor 170 may include or be positioned with one or more of the driver circuitry 120, the communication module 122, and/or the memory device 126 as described above for FIG. 9. In certain embodiments, the illumination device may further comprise the power source 124 as previously described for FIG. 9. In certain applications, the illumination device 168 may be used with and/or include the scalp covering 148 as described above for FIG. 12.

It is contemplated that any of the foregoing aspects, and/or various separate aspects and features as described herein, may be combined for additional advantage. Any of the various embodiments as disclosed herein may be combined with one or more other disclosed embodiments unless indicated to the contrary herein.

Those skilled in the art will recognize improvements and modifications to the preferred embodiments of the present disclosure. All such improvements and modifications are considered within the scope of the concepts disclosed herein and the claims that follow.

1. A phototherapy device for delivering light emissions to a scalp of a patient, the phototherapy device comprising:
a flexible substrate comprising a proximal surface and a distal surface that is opposite the proximal surface, the flexible substate being configured for positioning along a scalp of a user such that the proximal surface is closer to the scalp than the distal surface;
an array of light-emitting devices on the proximal surface;
driver circuitry configured to drive the array of light-emitting devices; and
a data capture device on the proximal surface of the flexible substrate, the data capture device configured to capture data of the scalp.

2. The phototherapy device of claim 1, further comprising a proximity sensor on the proximal surface of the flexible substrate.

3. The phototherapy device of claim 2, wherein the data capture device is configured to initiate capturing the data based on a signal from the proximity sensor.

4. The phototherapy device of claim 1, wherein the data capture device is a camera and the data comprises one or more images of the scalp.

5. The phototherapy device of claim 4, wherein the data comprises a progression of images configured to be captured as the camera is moved closer to the scalp.

6. The phototherapy device of claim 5, further comprising a flexible optic associated with the camera, wherein the flexible optic is configured to compress when the flexible optic contacts the scalp.

7. The phototherapy device of claim 6, wherein the camera is configured to initiate image capturing when the flexible optic contacts the scalp.

8. The phototherapy device of claim 1, wherein the data capture device is one of a plurality of data capture devices on the proximal surface of the flexible substrate.

9. The phototherapy device of claim 8, wherein the plurality of data capture devices comprises a first camera configured to capture a near field image of the scalp during use, and a second camera configured to capture a far field image of the scalp during use.

10. The phototherapy device of claim 1, further comprising a parting structure configured to create a part line in hair of the user.

11. The phototherapy device of claim 10, wherein the parting structure comprises a fan.

12. The phototherapy device of claim 11, wherein the parting structure comprises a comb feature.

13. The phototherapy device of claim 1, further comprising a scalp covering configured to conform to the scalp.

14. The phototherapy device of claim 13, wherein the scalp covering comprises a mesh structure to permit passage of light from the array of light-emitting devices.

15. The phototherapy device of claim 14, wherein the scalp covering comprises a window within a field of view of the data capture device.

16. The phototherapy device of claim 15, wherein the window comprises an antireflective material.

17. The phototherapy device of claim 1, wherein the array of light-emitting devices is configured to provide light to induce a biological effect for the scalp, the biological effect comprising at least one of vasodilation, inflammation reduction, nitric oxide generation, nitric oxide release, an antimicrobial effect, and treatment of hair loss.

18. A phototherapy device for delivering light emissions to a scalp of a patient, the phototherapy device comprising:
an array of light-emitting devices;
driver circuitry configured to drive the array of light-emitting devices; and
a data capture device configured to capture data of the scalp of a user, a position of the camera being adjustable relative to a position of the array of light-emitting devices.

19. The phototherapy device of claim 18, wherein the position of the data capture device is configured for manual adjustment.

20. The phototherapy device of claim 18, wherein the position of the data capture device is configured for automated adjustment.

21. The phototherapy device of claim 18, further comprising a flexible substrate comprising a proximal surface and a distal surface that is opposite the proximal surface, the flexible substate being configured for positioning along the scalp such that the proximal surface is closer to the scalp than the distal surface, wherein the array of light-emitting devices is on the proximal surface.

22. The phototherapy device of claim 21, further comprising a movement channel for the data capture device, the movement channel being formed through the flexible substrate.

23. The phototherapy device of claim 21, further comprising:
a cap comprising a cap proximal surface and a cap distal surface that is opposite the cap proximal surface, the cap proximal surface being positioned closer to the flexible substrate than the cap distal surface; and
a focus adjustment structure configured to provide focus control of the data capture device, the focus adjustment structure being accessible from the cap distal surface.

24. The phototherapy device of claim 18, further comprising a parting structure configured to create a part line in hair of the user.

25. The phototherapy device of claim 18, wherein the array of light-emitting devices is configured to provide light to induce a biological effect for the scalp, the biological effect comprising at least one of vasodilation, inflammation reduction, nitric oxide generation, nitric oxide release, an antimicrobial effect, and treatment of hair loss.

26. A phototherapy device for delivering light emissions to a scalp of a patient, the phototherapy device comprising:
an array of light-emitting devices;
driver circuitry configured to drive the array of light-emitting devices; and
a camera configured to capture one or more images of the scalp of a user via reflection from a mirror.

27. The phototherapy device of claim 26, further comprising a cap with an opening positioned to expose a portion of the scalp, wherein the mirror is positioned proximate the opening.

28. The phototherapy device of claim 27, wherein the mirror and camera reside within a housing on the cap.

29. The phototherapy device of claim 26, wherein the mirror is deformable.

30. The phototherapy device of claim 26, wherein the mirror forms a concave surface relative to the camera.

31. The phototherapy device of claim 26, further comprising a parting structure configured to create a part line in hair of the user.

32. The phototherapy device of claim 26, wherein the array of light-emitting devices is configured to provide light to induce a biological effect for the scalp, the biological effect comprising at least one of vasodilation, inflammation reduction, nitric oxide generation, nitric oxide release, an antimicrobial effect, and treatment of hair loss.

33.-55. (canceled)
